(19) United States
(12) Patent Application Publication
(10) Pub. No.: US 2026/0072045 A1
(43) Pub. Date: Mar. 12, 2026

(54) METHOD FOR DETECTING LYSOSOMAL STORAGE DISEASE BIOMARKERS AND KITS FOR PERFORMING THE METHOD

(71) Applicants: Universiteit Antwerpen, Antwerpen (BE); Universitair Ziekenhuis Antwerpen, Edegem (BE)

(72) Inventors: Amber VAN BAELEN, Wilrijk (BE); Stijn VERHULST, Wilrijk (BE); Francois EYSKENS, Edegem (BE)

(73) Assignees: Universiteit Antwerpen, Antwerpen (BE); Universitair Ziekenhuis Antwerpen, Edegem (BE)

(21) Appl. No.: 18/869,452

(22) PCT Filed: Jun. 1, 2023
(86) PCT No.: PCT/EP2023/064689

§ 371 Date: Nov. 26, 2024

(30) Foreign Application Priority Data
Jun. 1, 2022 (EP) 22176830.2
Mar. 21, 2023 (EP) 23163262.1

(51) Int. Cl. G01N 33/92

(52) U.S. Cl. CPC G01N 33/92; G01N 2405/10; G01N 2458/15; G01N 2800/04

(57) ABSTRACT

The present invention provides methods for detecting multiple biomarkers indicative for lysosomal storage diseases from a dried blood spot. In particular, the present invention provides a method that is suited for detecting multiple biomarkers, each indicative for the presence of a distinct lysosomal storage disease in a subject, based on a single sample preparation procedure. In particular, the method allows simultaneous extraction of different biomarkers such as Lyso-Gb1 (GlcSph). Lyso-Gb3, and others from a dried blood spot sample. The invention further provides a kit of parts comprising means for conducting the methods subject of the invention. Finally, the invention provides a set of reference ranges for Lyso-Gb1 (GlcSph) and Lyso-Gb3 in healthy subjects starting from dried blot spot samples.

### FIELD OF THE INVENTION

The present invention relates broadly to the field of molecular diagnostics and sample preparation for molecular diagnostics, particularly in a context of detecting multiple biomarkers each indicative for one or more lysosomal storage diseases. The invention further relates to kits of parts that provide a subject with the components for conducting the methods described herein.

### BACKGROUND OF THE INVENTION

Lysosomal storage diseases are a group of heterogeneous diseases caused by specific mutations affecting genes that encode either the lysosomal enzymes required for the degradation of a wide range of complex macromolecules, or the specific transporters that export the degradation products thereof out of the cell. The symptoms associated with lysosomal storage diseases vary depending on the particular disorder and age of onset and include developmental delay, movement disorders, seizures, dementia, deafness, blindness, enlarged organs such as liver and spleen, pulmonary problems, cardiac problems, and abnormal bone growth. Gaucher disease and Fabry disease are two of the most common lysosomal storage diseases, classified under the sphingolipidoses. The deficiency of β-glucocerebrosidase and α-galactosidase, respectively, results in the accumulation of the corresponding substrates giving rise to cellular and organ dysfunction.

Gaucher's disease (MIM606463) has an incidence in the general population that varies between 0.4-5.8/100,000 inhabitants but has been reported to be much more prevalent in certain populations such as the Ashkenazi Jewish population with an incidence of 1/800-1000. Beta-glucocerebrosidase is responsible for the degradation of the glycosphingolipid glucosylceramide (GlcCer), also known as glucocerebroside. Deficiency prevents the splicing of glucocerebroside into glucose and its fatty acyl moiety, ceramide, and results in the accumulation of glucosylceramide (Gb1) and the deacylated form (Lyso-) glucosylsphingosine (Lyso-Gb1, alternatively abbreviated in the art as “GlcSph”). Gb1 is only found intracellularly whereas Lyso-Gb1 can also be detected in blood and plasma due to its solubility in water. The deficiency is caused by a mutation in the GBA1 gene, located on chromosome 1 (1q21). Gb1 and Lyso-Gb1 accumulate in the lysosomes of the macrophages, known as Gaucher cells. As is the case in most lysosomal storage diseases, the clinical picture is more a phenotypic continuum of disease.

Fabry disease (MIM 301500) has been reported to occur with an incidence in the general population of ⅛ 454-117,000 live male births. However, based on data from newborn screening, including classic and late-onset disease in both males and females, the prevalence of this disease is higher. It is caused by an X-linked mutation of the GLA-gene, which impairs the normal function of α-galactosidase A, an enzyme responsible for hydrolysis of the final alpha galactosyl moiety of glycosylated lipids and proteins. Alpha-galactosidase A deficiency causes the accumulation of Globotriaosylceramide (Gb3) and its derivative Globotriosyl-sphingosine (Lyso-Gb3). Due to the progressive accumulation of glycosphingolipids in plasma, urine and lysosomes, diverse symptoms appear including heart and kidney failure, cerebrovascular disease, skin disorders and premature death.

In Gaucher disease and Fabry disease, the majority of patients gradually develop disease manifestations during childhood, but the diagnosis is mostly delayed until adulthood. A swift diagnosis is often hampered by the rare nature of lysosomal storage diseases in general and their diverse clinical presentation associated with a rather poor awareness among clinicians. The delayed diagnosis results in a delayed treatment, which is associated with increased disease complications. However, in case of an early diagnosis, preferably before puberty, existing therapy is able to improve the outcome of the disease.

Recent research showed that the detection of the deacylated forms of Gb1 and Gb3, Lyso-Gb1 (Gaucher disease) and Lyso-Gb3 (Fabry disease), in plasma and whole blood, have become a valuable and useful tool both in the diagnostic procedure as in the follow-up. Contrary to other suggested biomarkers such as Chitotriosidase and CCL18, Lyso-Gb1 and Lyso-Gb3 are specific for the Gaucher disease and Fabry disease, respectively. Several methods have been developed to measure either Lyso-Gb1 or Lyso-Gb3 in tissues, plasma, serum or urine (e.g., Hamler et al., Anal Chem. 2017: Sidhu et al., Biomed Chromatogr, 2018). Recently research has expanded towards the analysis of these biomarkers in dried blood spots. A dried blood spot sample offers several advantages such as the case of sampling, and stability of the compounds of interest during transport and storage (Cozma et al. Int J Mol Sci, 2020). To date, very little data about the detection of either biomarker in dried blood spot samples are available, and reference ranges for these biomarkers that would allow a fast diagnosis (and by extension any clinical relevant interpretation) for lysosomal storage diseases such as Fabry disease and Gaucher disease are non-existent.

There is therefore an unmet need in the field for novel and innovative screening methods that can assist with the early and specific detection of a lysosomal storage disease, which would greatly improve the diagnosis and early treatment of these patients.

### SUMMARY OF THE INVENTION

As evidenced by the examples enclosed herewith, the inventors have found a method that allows the rapid simultaneous extraction and detection of multiple lysosomal storage disease biomarkers from a patient sample without the necessity to conduct a dedicated method for each biomarker that is to be assessed (i.e., by means of a single sample processing procedure). The hereby presented method does not rely on different processing means for an individual biomarker, and thus greatly reduces the sample processing complexity and associated costs, while markedly increasing throughput possibilities for persons or automated machinery conducting the method. Since the method can be readily performed on dried blood spot samples, this enables a more convenient storage and transportation of the samples before processing. The inventors have found that the method developed allows for extraction and detection of lysosomal storage disease biomarkers such as Fabry disease and Gaucher disease by a single sample processing protocol. The present finding thus improves the diagnosis of patients having lysosomal storage diseases by allowing easy identification of multiple lysosomal storage diseases enabling treatment in an early stage of diseases included. The use of dried blood spot samples as input material for the method ensures a convenient manner to collect the necessary samples. For example, the present method allows that a sample can be taken less invasively and possibly from home, without the need for a hospital visit.

Accordingly, a first aspect of the invention provides in an in vitro method for detecting multiple biomarkers indicative for lysosomal storage diseases in a sample of a patient in one analysis, which comprises providing a dried blood spot sample of a patient and performing an extraction step and centrifugation step which allows simultaneous extraction of different biomarkers and detection of said biomarkers in the extract so obtained.

More particularly, a first aspect of the invention provides in an in vitro method for detecting multiple biomarkers indicative for lysosomal storage diseases in a sample of a patient, wherein the method comprises:
i) an extraction step of a dried blood spot sample of said patient or a portion thereof in an extraction solution comprising about 35% to about 65% dimethyl sulfoxide (DMSO) and about 35% to about 65% methanol (v/v);
ii) a centrifugation step comprising centrifugation of the sample obtained in step i) and collecting the supernatant;
iii) a detection step comprising detection of at least two biomarkers in the supernatant of step ii) each indicative for a distinct lysosomal storage disease.

In particular embodiments of the methods described above, the extraction solution comprises of from 40% to 60% DMSO and of from 40% to 60% methanol. In particular embodiments, the extraction solution comprises, consists essentially, or consists of about 50% DMSO and about 50% methanol.

In particular embodiments additionally or alternatively, a portion of a dried blood spot sample corresponding to from about 5 μl to 25 μl blood is incubated in the extraction solution, preferably a portion of a dried blood spot sample corresponding to about 10 μl is incubated in the extraction solution, more preferably a portion of a dried blood spot sample corresponding to 9.3 μl of blood is incubated in the extraction solution.

In particular embodiments additionally or alternatively, the extraction step comprises agitation of the dried blood spot sample, preferably wherein the extraction step comprises agitation on a vibration plate of the sample for at least 5 minutes. In embodiments, the extraction step comprises incubating the dried blood spot sample in the extraction solution for at least 10 minutes, preferably for at least 20 minutes.

In particular embodiments irrespective of the features detailed above, the extraction step is performed at a temperature of from about 18° C. to about 45° C., preferably at a temperature of about 37° C.

In particular embodiments of any one of the embodiments of the method described herein, the centrifugation step comprises centrifugation of the sample obtained in step i), preferably the centrifugation step comprises centrifugation of the sample obtained in step i) for at least 5 minutes, preferably for at least 7.5 minutes, more preferably for at least 10 minutes. In particular embodiments, the sample obtained in step i) is centrifuged for at least 5 minutes at from 2500 g to 15000 g, preferably for at least 5 minutes at 5250 g. In particular embodiments, the sample obtained in step i) is centrifuged at a temperature of about 15° C. to about 45° C., preferably at about 20° C.

In particular embodiments of the methods as described herein, the extraction solution used in step i) further comprises an internal standard, preferably wherein the extraction solution used in step i) further comprises an internal standard that comprises one or more stable isotope labeled reference biomarkers (i.e, one or more stable isotope labeled reference counterpart of the biomarkers to be detected).

The method as described herein can be a method for detecting a lysosomal storage disease selected from the group consisting of: sphingolipidosis, oligosaccharidosis, mucopolysaccharidosis, neuronal ceroid lipofuscinosis, sialic acid disorders, mucolipidosis, cholesteryl ester storage disease, glycogen storage disorders, and cystinosis, preferably the method is a method for detecting Gaucher disease and Fabry disease, most preferably the method is indicative for Gaucher disease, Fabry disease, and acid sphingomyelinase deficiency. In particular embodiments, the method is a method for discriminating between the different lysosomal storage diseases, more particularly between Gaucher disease and Fabry disease, i.e., a method of determining whether the patient is suffering from Gaucher disease or Fabry disease.

In the methods described herein each biomarker can be indicative for a lysosomal storage disease selected from the group consisting of: sphingolipidosis, oligosaccharidosis, mucopolysaccharidosis, neuronal ceroid lipofuscinosis, sialic acid disorders, mucolipidosis, cholesteryl ester storage disease, glycogen storage disorders, and cystinosis, preferably wherein at least one biomarker is indicative for Gaucher disease and at least one biomarker is indicative for Fabry disease, more preferably wherein at least one biomarker is indicative for Gaucher disease, at least one biomarker is indicative for Fabry disease, and optionally at least one biomarker that is indicative for acid sphingomyelinase deficiency (i.e. Niemann-Pick disease (type A, A/B, and/or B), or Krabbe discase.

In particular embodiments of the methods as envisaged herein, at least one biomarker is a sphingolipid or a molecule derived from a sphingolipid. Optionally, each biomarker is a sphingolipid or a molecule derived from a sphingolipid. Optionally, each biomarker is a sphingolipid. Optionally, each biomarker is a lyso-sphingolipid.

In further particular embodiments, one or more of the plurality of biomarkers are selected from the group consisting of: glucosylsphingosine (Lyso-Gb1, interchangeably referred to as GlcSph), globotriaosylsphingosine (Lyso-Gb3), lysosphingomyelin and lyso-galactosylsphingosine. In particular embodiments, each of the plurality of biomarkers are selected from the group consisting of: glucosylsphingosine (Lyso-Gb1, interchangeably referred to as GlcSph), globotriaosylsphingosine (Lyso-Gb3), lysosphingomyelin-509, and lysosphingomyelin (Lyso-SPM). In particular embodiments, a first biomarker is glucosylsphingosine (Lyso-Gb1, interchangeably referred to as GlcSph) and a second biomarker is globotriaosylsphingosine (Lyso-Gb3).

Optionally, in the methods described herein above, the detection step comprises detection of at least one biomarker by mass spectrometry, preferably tandem mass spectrometry. In preferable embodiments, the detection step comprises detection of at least two biomarkers by tandem mass spectrometry. In more preferably embodiments, the detection step comprises detection of each of the biomarkers by tandem mass spectrometry. 20 Additionally or alternatively, the detection step of the method comprises a targeted tandem mass spectrometry assay. In preferred embodiments, the detection step of the method is a targeted tandem mass spectrometry assay selected from the group consisting of: Selected Reaction Monitoring (SRM), Multiple Reaction Monitoring (MRM), or Parallel Reaction Monitoring (PRM). In more preferable embodiments, the targeted tandem mass spectrometry assay comprises measurement of a parent Lyso-Gb1 ion and a parent Lyso-Gb3 ion. In most preferable embodiments, the targeted tandem mass spectrometry assay comprises measurement of a parent Lyso-Gb1 ion, a parent Lyso-Gb3 ion, and at least one daughter Lyso-Gb1 ion and one daughter Lyso-Gb3 ion.

In particular embodiments of the methods as described herein above, the targeted tandem mass spectrometry assay comprises measurement of a parent Lyso-Gb1 ion, a parent Lyso-Gb3 ion, and a parent Lyso-SPM ion. More particularly, the targeted tandem mass spectrometry assay may comprise measurement of a parent Lyso-Gb1 ion at 462.2 to 462.3 m/z and a parent Lyso-Gb3 ion at 786.2 to 786.8 m/z. In preferred embodiments, the targeted tandem mass spectrometry assay comprises measurement of a parent Lyso-Gb1 ion at 462.29 m/z, more specifically 462.294 m/z. In alternative and/or complementary preferred embodiments, the targeted tandem mass spectrometry assay comprises measurement of a parent Lyso-Gb3 ion at 786.3 m/z or at 786.4 m/z, more specifically 786.392 m/z.

In further particular embodiments, the targeted tandem mass spectrometry assay comprises measurement of a daughter Lyso-Gb1 ion at 282.0 m/z to 282.5 m/z and a daughter Lyso-Gb3 ion at 282.0 m/z to 282.5 m/z. In particular embodiments, the targeted tandem mass spectrometry assay comprises measurement of a daughter Lyso-Gb1 ion at 282.3 m/z and a daughter Lyso-Gb3 ion at 282.3 m/z. In preferred embodiments, the targeted tandem mass spectrometry assay comprises measurement of a parent Lyso-Gb1 ion at 462.2 to 462.3 m/z, a parent Lyso-Gb3 ion at 786.2 to 786.8 m/z, a daughter Lyso-Gb1 ion at 282.0 m/z to 282.5 m/z and a daughter Lyso-Gb3 ion at 282.0 m/z to 282.5 m/z.

Also envisaged are particular embodiments of the methods herein, wherein the targeted tandem mass spectrometry assay comprises measurement of a parent Lyso-Gb1 ion at 462.294 m/z, a parent Lyso-Gb3 ion at 786.392 m/z, a daughter Lyso-Gb1 ion at 282.3 m/z and a daughter Lyso-Gb3 ion at 282.3 m/z.

Optionally, in these methods, the obtained Lyso-Gb1 value is compared with a reference range to determine whether a subject has Gaucher Disease, or to determine whether a treatment is effective in a subject diagnosed to have Gaucher Disease. Optionally, the reference range as present in a healthy subject (in the present context a subject not having Gaucher Disease) is age dependent. For example, a suitable reference range for a healthy subject up to about 4 years old may be from 0.282 ng/ml to 6.890 ng/ml; may be from 0.235 ng/ml to 5.409 ng/ml for a healthy subject from about 4 years old to about 12 years old; may be from 0.237 ng/ml to 6.385 ng/ml for a healthy subject from about 12 years old to about 18 years old; may be from 0.272 ng/ml to 7.111 ng/ml for a healthy subject from about 18 years old to about 40 years old; may be from 0.319 ng/ml to 6.392 ng/ml for a healthy subject from about 40 years old to about 60 years old; may be from 0.317 ng/ml to 4.928 ng/ml for a healthy subject over 60 years old.

Additionally or alternatively, the obtained Lyso-Gb3 value is compared with a reference range to determine whether a subject has Fabry Disease, or to determine whether a treatment is effective in a subject diagnosed to have Fabry Disease. Optionally, the reference range as present in a healthy subject (in the present context a subject not having Fabry Disease) is age dependent. For example, a suitable reference range for a healthy subject up to about 4 years old may be from 0.0441 ng/ml to 1.952 ng/ml; may be from 0.223 ng/ml to 2.326 ng/ml for a healthy subject from about 4 years old to about 12 years old; may be from 0.138 ng/ml to 2.014 ng/ml for a healthy subject from about 12 years old to about 18 years old; may be from 0.145 ng/ml to 1.971 ng/ml for a healthy subject from about 18 years old to about 40 years old; may be from 0.085 ng/ml to 2.014 ng/ml for a healthy subject from about 40 years old to about 60 years old; may be from 0.118 ng/ml to 1.774 ng/ml for a healthy subject over 60 years old. Preferably, a suitable reference range for a healthy female subject up to about 4 years old may be from 0.0441 ng/ml to 1.518 ng/ml and/or for a healthy male subject up to about 4 years old may be from 0.057 ng/ml to 1.952 ng/ml. Preferably, a suitable reference range for a healthy female subject from about 4 years old to about 12 years old may be from 0.262 ng/ml to 2.217 ng/ml and/or for a healthy male subject from about 4 years old to about 12 years old may be from 0.223 ng/ml to 2.326 ng/ml. In particular embodiments, the method further comprises a step of detecting an enzyme (and/or a defective enzyme) causative of a lysosomal storage disease and/or a reduction or absence of enzymatic activity of an enzyme causative of a lysosomal storage disease in the sample. In preferred embodiments, the method further comprises a step of detecting enzymatic activity of at least one enzyme selected from the group consisting of: glucocerebrosidase, alfa-galactosidase, and acid sphingomyelinase.

In a further related aspect, the invention is directed to a kit of parts for detecting multiple biomarkers indicative for lysosomal storage diseases from a dried blood spot sample, such as the methods described herein above, wherein the kit comprises:
an extraction solution comprising 35% to 65% DMSO and 35% to 65% methanol (v/v);
an internal standard comprising at least two stable isotope labeled reference counterparts of said multiple biomarkers (wherein each of said biomarkers is indicative for a lysosomal storage disease); and
a set of instructions for performing the method subject of the invention and (i.e. as described in any embodiment of the present disclosure). In particular embodiments, the kit of parts comprises as a first reference biomarker stable isotope labeled Lyso-Gb1 and as a second reference biomarker stable isotope labeled Lyso-Gb3. In further particular embodiments, the kit of parts comprises as first reference biomarker stable isotope labeled Lyso-Gb1, as a second reference biomarker stable isotope labeled Lyso-Gb3, and as a third reference biomarker stable isotope labeled Lyso-SPM.

The above and further aspects and preferred embodiments of the invention are described in the following sections and in the appended claims. The subject matter of the appended claims is hereby specifically incorporated in this specification.

### BRIEF DESCRIPTION OF THE FIGURES

FIG. 1: Representation of the linearity based on the 7 standard values for each biomarker. Analyzation by comparing the spiked concentrations with the measured concentrations. A: Bland-Altman LGB1. B: Bland Altman LGB3. C: LGB1. D: LGB3. Y-axis of C and D: Measured concentration (ng/ml). X-axis of C and D: Theoretic concentration (ng/ml).

FIG. 2: A Boxplot analysis of the measured QC values of Lyso-Gb1.

FIG. 3: A Boxplot analysis of the measured QC values of Lyso-Gb3.

FIG. 4: Chromatographic printout of the detection of QC1 of Lyso-Gb1 representing the signal-to-noise ratio for the analysis of the LOQ. Y-axis:Intensity, cps.

FIG. 5: Chromatographic printout of the detection of QC1 of Lyso-Gb3 representing the signal-to-noise ratio for the analysis of the LOQ. Y-axis:Intensity, cps.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . , and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥25, ≥26 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

Given that lysosomal storage diseases are rare inherited metabolic disorders and can manifest themselves by means of a diverse set of clinical presentations presents a challenge for (general) medical practitioners (Mehta et al., Intern Med J, 2020; Bernardes et al., Rev Assoc Med Bras, 2020). However, a fast and specific diagnosis is important due to disease complications that occur at later disease stages (Wenger et al., Genet Med, 2002). Early detection (preferably during childhood) and starting specific therapy is therefore of the utmost importance for patients.

Despite the clear need, advances in diagnostic methods and any multiplexing thereof remain scarce. The present inventors have identified a single method that is suitable for detecting different biomarkers that are each indicative for a distinct lysosomal storage disease in a simple way and in a single sample detection step, for example in a single mass spectrometry detection step (i.e. run). Moreover, due to the capability of the method to use dried blood spot samples as input material, biomarker detection can conveniently be combined with additional enzymatic assays. The method presented herein provides the first instance wherein multiple lysosomal storage disease biomarkers such as Lyso-Gb1 and Lyso-Gb3 are detected in an extract of a single sample, with one processing method. Hence, the present invention pushes the boundaries of lysosomal storage disease diagnostics.

Accordingly, the invention provides in an in vitro method for detecting multiple biomarkers indicative for lysosomal storage diseases in a sample of a patient, which comprises providing a dried blood spot sample of said patient, performing an extraction step and centrifugation step which allows simultaneous extraction of different biomarkers, and detection of said biomarkers in the extract so obtained.

Thus, in a first aspect the present invention provides a method for detecting multiple biomarkers indicative for lysosomal storage diseases such as sphingolipidoses from a dried blood spot sample, wherein the method comprises:
i) an extraction step of the dried blood spot sample or a portion thereof in an extraction solution comprising of from 35% to 65% dimethyl sulfoxide (abbreviated herein by “DMSO”) and of from 35% to 65% methanol (v/v);
ii) a centrifugation step comprising centrifugation of the sample obtained in step i) and collecting the supernatant;
iii) a detection step comprising detection of at least two biomarkers in the supernatant of step ii) each indicative for a distinct lysosomal storage disease.

The expression “multiple biomarkers” as used herein, which may be used interchangeably with for example “a plurality of biomarkers” refers to at least 2 biomarkers, but is not limited to exactly 2 biomarkers. Hence, a plurality of biomarkers or multiple biomarkers may broadly refer to 2 biomarkers, but equally to 3, 4, 5, 6, 7, 8, 9, 10, or more than 10 biomarkers. The term may equally refer to at least 2, at least 3, at least 4, at least 5, at least 6, at least 7, at least 8, at least 9, at least 10, or more than 10 biomarkers. Preferably, the plurality of biomarkers where to reference is made in the present context is to be interpreted as “at least 2 biomarkers”.

The term “biomarker”, often indicated in the art by the term “marker”, is widespread in the art and commonly broadly denotes a biological component or a biological molecule, more particularly an endogenous biological component or molecule, or a detectable portion thereof, whose qualitative and/or quantitative evaluation in a tested subject, such as by means of evaluating a biological sample from the subject, is predictive or informative with respect to one or more aspects of the tested subjects phenotype and/or genotype. The term “biomarker” therefore encompasses any physical form of the biomarkers including proteins, polypeptides, peptides, nucleic acids, and any metabolic products thereof. In the present context. “a plurality of biomarkers” and “multiple biomarkers” may be used interchangeably with related terms and expressions such as but not limited to “a biomarker signature”, “a set of biomarkers”, or “a biomarker collection” which each indicate the presence and/or level of a combination of biomarkers, said combination being characteristic for a discrete condition, stage of condition, subtype of condition or a prognosis for a discrete condition, stage of condition, subtype of condition.

The method and kits described herein aim to provide a subject and/or medical practitioner with information with respect to said subject having a certain lysosomal storage disease. Hence, it is appropriate to alternatively express the method as “a method of diagnosing”. “a method of molecular profiling”, and the likes. “Molecular profiling” broadly relates to the practice of identification of one or more individual profiles that allow for more informed and effective personalized treatment options, which can result in improved patient care, enhanced treatment outcome, and improved treatment efficacy as is known to a skilled person. Moreover, molecular profiling indirectly improves follow up of patients that are receiving or have received treatment.

The method described herein and associated kits are suitable to provide a single protocol for detection and quantitation of multiple biomarkers based on a single unitary sample processing workflow. Thus, the method described herein allows for predicting and/or deducting the disease status of a subject starting from a dried blood spot sample. A subject is considered to have a lysosomal storage disease when the detection step of the method indicates that a certain threshold value for one of the tested biomarkers is exceeded.

In certain embodiments of the method as described herein, the threshold value is an arbitrary value determined by experts in the field, or determined by the person or automated machine conducting the method. In other embodiments, the subject whose dried blood spot sample is analysed is considered to have a lysosomal storage disease when a certain value obtained from (i.e. derived from) detecting the reference biomarker level of the internal standard is exceeded. Hence, the internal standard does not necessarily need to provide an absolute value whereby a biomarker level of a subject (measured by performing the method described herein on a dried blood spot sample obtained from said subject) subject exceeding said value is considered to have a lysosomal storage disease, but may instead provide a calibration point for arriving at the actual biomarker level in the dried blood spot sample. Optionally, the subject whose dried blood spot sample or portion thereof is used as input material in the method described herein is considered to have a lysosomal storage disease when the detection step of the method indicates that a certain ratio of a biomarker of a subject to a reference biomarker in the internal standard.

Alternatively worded, the subject whose dried blood spot sample or portion thereof is used as input material in the method described herein is considered to have a lysosomal storage disease when one biomarker that is detected in the detection step has a differential (expression) level when compared to a reference value determined by the internal standard or determined a priori.

Optionally, the reference level(s) or reference value(s) may be derived from a digital database, such as a computer database. Said database may comprise a collection of (reference) biological samples whereof the level of the biomarkers of interest have been determined. Alternatively, the database may generate suitable reference levels upon input by a user of one or more subject characteristics, such as age, gender, and/or ethnicity.

Terms such as “different level” and “differential expression level” imply a measurable difference in expression level (i.e, the extent to which an analyte is present in an analysed sample and therefore a proxy for the extent to which said analyte is present in the subject from which said sample is obtained from), and evidently implies statistical significance of the difference between the expression level of the biological sample and the reference biological sample, or at least a trend that may be deducted upon analysing the expression levels. By means of illustration and not limitation, a suitable threshold for attributing statistical significance in expression level is a 2 log change characterised by a p value <0.001 and a false discovery rate (FDR)<0.05.

Alternatively, the “different (expression) level” may be expressed as a “deviation of expression level” of one or more of the analysed biomarkers in a sample (in the context of the present invention obtained from a subject suspected of having a lysosomal storage disease or a subject being screened for the presence of a lysosomal storage disease) when compared to a reference biological sample (e.g., a healthy subject known to not have said lysosomal storage disease) or internal standard. A “deviation” of a first expression level of a biomarker in the sample obtained from the subject from a second expression level of said biomarker in the internal standard may generally encompass any direction and any extent of alteration.

Without limitation, a deviation may encompass an absolute difference of the expression levels or of one or more biomarkers of e.g., at least about 10%, e.g., of at least about 20%, of at least about 30%, e.g., of at least about 40%, of at least about 50%, e.g., of at least about 60%, of at least about 70%, e.g., of at least about 80%, of at least about 90%, e.g., of at least about 95%, such as of at least about 96%, 97%, 98%, 99% or even of 100%, preferably at least about 70%, more preferably at least about 80%, even more preferably at least about 90%, relative to the reference biomarker level for said one or more biomarkers as obtained from an internal standard and/or a reference biological sample (e.g., a subject that is known to not have the, or any, lysosomal storage disease).

In the context of the present invention, a deviation refers to a statistically significant observed alteration in the level of one or more biomarkers included in the method subject of the present invention. For example, a deviation may refer to an observed alteration or increase, which falls outside of error margins of reference levels obtained from an internal standard and/or a reference biological sample (as expressed, for example, by standard deviation (SD) or standard error (SE), or by a predetermined multiple thereof, e.g., ±1×SD or ±2×SD or ±3×SD, or ±1×SE or ±2×SE or ±3×SE). Deviation or reduction may also refer to a value falling outside of a reference range defined by the expression levels measured in multiple reference biological samples (for example, outside of a range which comprises ≥40%, ≥50%, ≥60%, ≥70%, ≥75%, ≥80%, ≥85%, ≥90%, ≥95%, or even ≥100% of expression levels measured in said reference biological samples).

The method described herein uses dried blood spot samples as input material (i.e. starting material). In the context of the present invention, each biomarker that is detected by the method described herein is indicative for the presence of a lysosomal storage disease in the sample, and thus ultimately for the disease status of a patient whose blood was applied to the spot. Thus, the method defined herein provides information with respect to the disease status of a subject for at least two different lysosomal storage diseases.

Terms such as “subject”, “patient”, and “individual” may be used interchangeably herein and refer to animals, preferably warm-blooded animals, more preferably vertebrates, and even more preferably mammals specifically including humans. Preferred subjects are human subjects including all genders and all age categories thereof. Both adult subjects, new-born subjects, and foetuses are intended to be covered by the term “subject”. Preferred subjects in the context of the invention are human subjects that are presented to a medical practitioner such as a physician with symptoms and signs indicative of a lysosomal storage disease or a group of lysosomal storage diseases. Preferred subjects in the context of the present invention are subjects of from 0 to 18 years of age. Optionally, the method described herein is a method used for new-born screening. Any references to certain proteins or genes throughout the present disclosure indicate human proteins or human genes unless explicitly stated otherwise.

The method described in the present disclosure thus represent a means for establishing that a subject has a lysosomal storage disease, i.e., establishing a diagnosis of a lysosomal storage disease. The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of one or more lysosomal storage diseases). As used herein, “diagnosis of” the diseases or conditions as taught herein in a subject may particularly mean that the subject has such, hence, is diagnosed as having such.

The method described in the present disclosure further represent a means for establishing a certain prognosis for a subject that has a lysosomal storage disease. In such embodiments, the status of the subject having a lysosomal storage disease may be established by means of, or as part of, a new born screening programs.

The term “lysosomal storage disease” as used herein encompasses any inherited disorder that is characterized by reduced or absent lysosomal enzyme activity. Alternatively worded, lysosomal storage diseases are inborn errors of metabolism characterized by the accumulation of substrates in various cells due to the defective functioning of lysosomes. Said substrates cause dysfunction of one or more organs where they accumulate and contribute to considerable morbidity and mortality. While different lysosomal storage disorders are characterised by different deficiencies in lysosomal enzyme function, (aberrant) accumulation of material in lysosomes is the common cause for each of these diseases. Specific lysosomal storage diseases preferred in the context of the methods and kit of parts described herein are described in detail further below. The term “lysosomes” is well known to a person skilled in the art and refers to a membrane-bound organelle in mammalian cells that contains hydrolytic enzymes capable to deteriorate numerous different biomolecules including peptides, nucleic acids, lipids, and carbohydrates. Lysosomes are further characterised by an average size of about 0.1 μm to 1.2 μm and generally have a pH of from about 4.5 to about 5. Detailed reviews have been published describing lysosomes and lysosomal function and are therefore part of the art (e.g. Ballabio and Bonifacino, Nat Rev Mol Cell Biol, 2020; and Bouhamdani et al., Front Cell Dev Biol, 2021).

The method described herein is tailored to use as input material a dried blood spot on filter paper (i.e., a dried blood spot sample), or portion thereof. A skilled person appreciates that a dried blood spot sample corresponds to a blood samples that is blotted and dried on filter paper, or directly applied to (i.e. collected on) and dried on filter paper. Particulars of dried blood spots have been described at numerous instances throughout the art (e.g., in Deep et al., Int J Pharm Sci Rev Res, 2012). The precise composition of the filter paper is not particularly limiting for the method described herein, which can essentially be performed starting from any substrate that is capable to retain a dried blood sample. A skilled person further appreciates that dried blood spots provide multiple advantages for sample collection and preservation when compared to traditional sampling methods including but not limited to whole blood collection, serum collection, or plasma sample collection. By means of illustration and not limitation, advantages include the less invasive nature of the sampling method (e.g., by means of a finger, toc, or heel prick), sample storage and case of transportation, reduced infection risks for persons handling the blood spot, and a reduced volume of sample that needs to be extracted from the subject.

The method described herein is an in vitro method. “In vitro” broadly refers to outside of, or external of the body of a subject. The present of a subject is therefore not essential for performing the method described herein. Each step of the method described herein is performed without any instance of physical interaction with the body of a subject.

“Extraction solution” as used herein refers to the solution whereto/wherein the dried blood spot sample or portion thereof is incubated in and optionally detaches the sample from the filter paper used as substrate for the dried blood spot sample. Preferably, the extraction solution substantially surrounds or surrounds the dried blood spot or portion thereof during the extraction step. The inventors have identified that an extraction solution comprising dimethyl sulfoxide (DMSO) and methanol is particularly effective to act as an extraction solution. The extraction solution used in the method described herein comprises of from about 35% to about 65% DMSO volume concentration and of from about 35% to about 65% methanol volume/volume percentage (v/v). Preferably, the extraction solution used in the method described herein comprises of from about 40% to about 60% DMSO and of from about 35% to about 60% methanol (v/v), more preferably of from about 45% to about 55% DMSO and of from about 35% to about 55% methanol (v/v), more preferably of from about 47.5% to about 52.5% DMSO and of from about 35% to about 52.5% methanol (v/v), most preferably about 50% DMSO and of from about 35% to about 50% methanol (v/v). Alternatively, the extraction solution used in the method described herein may comprise of from about 35% to about 60% DMSO and of from about 40% to about 60% methanol (v/v), preferably of from about 35% to about 55% DMSO and of from about 45% to about 55% methanol (v/v), more preferably of from about 35% to about 52.5% DMSO and of from about 47.5 to about 52.5% methanol (v/v), most preferably of from about 35% to about 50% DMSO and about 50% methanol (v/v). It is appreciated by a skilled person that incubation of a dry sample with an extraction solution results to an increase in weight and/or increase in volume of said sample.

Preferably, the extraction solution consists essentially of DMSO and methanol, preferably the extraction solution consists essentially of about 50% DMSO and 50% methanol (v/v). The extraction solution may consist of DMSO and methanol, preferably the extraction solution consist of about 50% DMSO and 50% methanol (v/v).

The terms “volume/volume percentage indicates the volume of liquid to the total volume of the formulation (i.e. mass fraction) with a denominator of 100. It is routinely expressed in the art as the ratio of the volume of a first component to the total volume of the solution multiplied by 100.

In preferred embodiments, a portion of a dried blood spot sample is used for incubation in the extraction solution. Optionally, the portion (i.e., fragment, part, piece, section, segment) of the dried blood spot sample used in the method described herein corresponds to a spotted blood volume of from about 1 to about 100 μl, preferably to a spotted blood volume of from about 2.5 μl to about 50 μl, more preferably to a spotted blood volume of from about 5 μl to about 25 μl, even more preferably of from 7.5 μl to about 20 μl, yet even more preferably of from about 8 μl to 15 μl, most preferably of from about 9 μl to about 12 μl. In a most preferred embodiment, the portion of the dried blood spot sample used in the method described herein corresponds to a spotted blood volume of about 9.3 μl. Optionally, the portion (i.e., fragment, part, piece, section, segment) of the dried blood spot sample used in the method described herein corresponds to at least one punch of about ⅛ inches (0.3175 cm), preferably at least two punches of about ⅛ inches (0.3175 cm), more preferably to at least three punches of about ⅛ inches (0.3175 cm).

By means of illustration and not limitation, a suitable device for reducing the size of the dried blood spot sample which therefore allows isolating a portion of the dried blood spot sample is a semi-automatic Punchers (PerkinElmer).

Preferably, the extraction step of the dried blood spot sample or a portion thereof in the extraction solution further comprises agitation of said sample. In the context of the present invention, the term “agitation” broadly refers to the action of putting something in motion by shaking and/or stirring. The exact agitation means and agitation period is not particularly limiting in the present context. Preferably, the agitation of the incubated sample is achieved by means of orbital shaking. More preferably, the agitation of the incubated sample is achieved by means of orbital shaking at a constant or essentially constant velocity. Alternatively, the agitation of the incubated sample is achieved by means of orbital shaking at variable velocity.

By means of illustration and not limitation, a suitable orbital shaker for use in the method described herein is a DELFIA PlateShake (PerkinElmer).

In certain embodiments, the extraction step has a duration of at least 2.5 minutes. Preferably, the extraction step has a duration of at least 5 minutes, more preferably at least 7.5 minutes, yet more preferably at least 10 minutes, even more preferably at least 15 minutes, most preferably at least 20 minutes. A skilled person appreciates that the term “at least x” is indicative of an open range corresponding to “of from x to ∞ (infinity)”. Alternatively, the ranges can be expressed by a defined upper limit, and the extraction step may therefore have a duration of from 2.5 minutes to 180 minutes, preferably of from 5 minutes to 120 minutes, more preferably of from 7.5 minutes to 90 minutes, yet more preferably of from 10 minutes to 75 minutes, even more preferably of from 15 minutes to 60 minutes, most preferably of from 20 minutes to 45 minutes.

The sample temperature during the extraction step is not particularly limiting for the present invention. Hence, the extraction step may be performed at room temperature (i.e. ambient temperature). Alternatively, the extraction step may be performed at a temperature of from about 15° C. to about 60° C., preferably of from about 18° C. to about 55° C., preferably of from about 20° C. to about 55° C., preferably of from about 20° C. to about 52° C., preferably of from about 22° C. to about 50° C., preferably of from about 25° C. to about 47.5° C., preferably of from about 28° C. to about 45° C., preferably of from about 30° C. to about 42° C., preferably of from about 32° C. to about 40° C., preferably of from about 34° C. to about 38° C., most preferably wherein the extraction step is performed at a temperature of about 37° C. In certain embodiments, the extraction steps and centrifugation steps may be characterised by incubation of the sample at different temperatures. For example, the extraction step may be performed at about 37° C., while the centrifugation step may be performed at room temperature (i.e. ambient temperature).

Preferred embodiments of the method have an extraction step wherein the dried blood spot sample in the extraction solution is incubated under agitation for a duration of at least 5 minutes at a temperature of from about 15° C. to about 60° C., preferably for a duration of at least 10 minutes at a temperature of from about 28° C. to about 45° C., more preferably for a duration of at least 15 minutes at a temperature of from about 32° C. to about 40° C., most preferably for a duration of at least 20 minutes at about 37° C. Alternative preferred embodiments of the method have an extraction step wherein the dried blood spot sample in the extraction solution is incubated under agitation for a duration of from 5 minutes to 180 minutes at a temperature of from about 15° C. to about 60° C., preferably for a duration of from 10 minutes to 75 minutes at a temperature of from about 28° C. to about 45° C., more preferably for a duration of from 15 minutes to 60 minutes at a temperature of from about 34° C. to about 38° C., most preferably for a duration of from 20 minutes to 45 minutes at about 37° C.

The centrifugation step comprises centrifugation of the sample obtained in step i) of the method. Preferably, the sample is centrifuged for at least 2.5 minutes at from about 2500 g to about 15000 g. more preferably, the sample is centrifuged for at least 5 minutes at from about 2500 g to about 15000 g, even more preferably the sample is centrifuged for at least 7.5 minutes at from about 2500 g to about 15000 g, most preferably the sample is centrifuged for at least 10 minutes at from about 2500 g to about 15000 g. Alternatively, the ranges can be expressed by a defined upper limit, and centrifugation may be performed for a duration of from 2.5 minutes to 180 minutes at from about 2500 g to about 15000 g. preferably for a duration of from 5 minutes to 75 minutes at from about 2500 g to about 15000 g, more preferably for a duration of from 7.5 minutes to 60 minutes at from about 2500 g to about 15000 g, most preferably for a duration of from 10 minutes to 45 minutes at from about 2500 g to about 15000 g.

By means of illustration and not limitation, a suitable centrifugation device for use in the method described herein is a Allegra x-15R device (Beckman Coulter).

Preferably, the centrifugation of the sample obtained in step i) of the method comprises centrifugation for at least 2.5 minutes at from about 2500 g to about 12500 g, preferably at from about 2500 g to about 10000 g, more preferably at from about 2500 g to about 7500 g, even more preferably at from about 3500 g to about 6500 g, yet even more preferably at from about 4500 g to about 5500 g, most preferably at about 5250 g.

In preferred embodiments, the centrifugation of the sample obtained in step i) of the method comprises centrifugation for at least 2.5 minutes at from about 2500 g to about 15000 g, preferably for at least 5 minutes at from about 2500 g to about 7500 g, more preferably for at least 7.5 minutes at from about 4500 g to about 5500 g, most preferably for at least 10 minutes at about 5250 g. A skilled person is aware of how to calculate g force starting from a certain round per minute (i.e., RPM) and diameter of a given centrifuge. By means of illustration and not limitation, 5250 g on an Allegra x-15R corresponds to 4750 RPM on the Allegra x-15R.

The sample temperature during centrifugation when performing the centrifugation step is not particularly limiting for the present invention. Hence, centrifugation may be performed at room temperature (i.e. ambient temperature). Alternatively, centrifugation may be performed at a temperature of from about 15° C. to about 45° C., preferably of from about 16° C. to about 45° C., preferably of from about 17° C. to about 42° C., preferably of from about 18° C. to about 40° C., preferably of from about 18° C. to about 37.5° C., preferably of from about 18° C. to about 35° C., preferably of from about 18° C. to about 32° C., preferably of from about 18° C. to about 28° C., preferably of from about 19° C. to about 26° C. preferably of from about 20° C. to about 25° C.

Preferred embodiments of the method described herein have a centrifugation step wherein the sample obtained in step i) of the method is centrifuged for a duration of at least 2.5 minutes at a temperature of from about 15° C. to about 45° C., preferably for a duration of at least 5 minutes at a temperature of from about 17° C. to about 42° C., more preferably for a duration of at least 7.5 minutes at a temperature of from about 18° C. to about 37.5° C., most preferably for a duration of at least 10 minutes at room temperature (i.e. ambient temperature), or about 20° C. Alternative preferred embodiments of the method are characterized by a centrifugation step comprising centrifugation for a duration of from 2.5 minutes to 180 minutes at a temperature of from about 15° C. to about 45° C., preferably for a duration of from 5 minutes to 75 minutes at a temperature of from about 17° C. to about 42° C., more preferably for a duration of from 7.5 minutes to 60 minutes at a temperature of from about 18° C. to about 37.5° C., most preferably for a duration of from 10 minutes to 45 minutes at room temperature (i.e. ambient temperature).

The term “supernatant” as described herein is indicative for the liquid situated above a solid residue after crystallization, precipitation, or centrifugation. In the context of the present invention, “supernatant” refers to the liquid situated above a solid residue after centrifugation unless specified otherwise. In certain embodiments, the supernatant of the sample obtained by centrifugation is collected by decantation of said sample (contained in a first container) into a different container. In alternative embodiments, the supernatant of the sample obtained by centrifugation is collected by aspiration. In yet alternative embodiments, the supernatant of the sample obtained by centrifugation is collected be pipetting. A skilled person appreciates that the manner of collecting the supernatant is not particularly limiting for the present invention.

Preferably, the method described herein is characterized by i) an extraction step of the dried blood spot sample or portion thereof for at least 10 minutes at 18° C. to 45° C. under agitation in an extraction solution comprising of from 40% to 60% DMSO and of from 40% to 60% of methanol; ii) a centrifugation step comprising centrifugation for 2.5 minutes at from 2500 g to 12500 g at 15° C. to 45° C. of the sample obtained in step i) and collecting the supernatant; and iii) a detection step comprising detection of at least two biomarkers in the supernatant of step ii) each indicative for a distinct lysosomal storage disease by means of mass spectrometry. More preferably, the method described herein is characterized by i) an extraction step of the dried blood spot sample or portion thereof for at least 15 minutes at of from 32° C. to 40° C. under agitation in an extraction solution comprising of from 45% to 55% DMSO and of from 45% to 55% of methanol: ii) a centrifugation step comprising centrifugation for 5 minutes at from 2500 g to 12500 g at 18° C. to 28° C. of the sample obtained in step i) and collecting the supernatant; and iii) a detection step comprising detection of at least two biomarkers in the supernatant of step ii) each indicative for a distinct lysosomal storage disease by means of mass spectrometry. Most preferably, the method described herein is characterized by i) an extraction step of the dried blood spot sample or portion thereof for about 20 minutes at 37° C. under agitation in an extraction solution comprising about 50% DMSO and about 50% of methanol: ii) a centrifugation step comprising centrifugation for at about 10 minutes at about 5250 g at about 20° C. of the sample obtained in step i) and collecting the supernatant; and iii) a detection step comprising detection of at least two biomarkers in the supernatant of step ii) each indicative for a distinct lysosomal storage disease by means of one or more targeted mass spectrometry assays.

Optionally, the extraction solution further comprises an internal standard. Alternatively, an internal standard is added to the dried blood spot sample during incubation with the extraction solution. Yet alternatively, an internal standard is added to the supernatant obtained after centrifugation of the sample obtained in step i) of the method described herein. Yet even more alternatively, the internal standard is added to the blood spot sample prior to drying and obtaining the dried blood spot sample. Yet even more alternatively, the internal standard is present on or in the filter papier prior to contacting the filter paper with the blood sample, hence prior to drying the blood spot sample and obtaining the dried blood spot sample. The internal standard may be a reference counterpart (i.e., version, form, presentation, moiety, molecule) of at least one biomarker part of the plurality of biomarkers each indicative for lysosomal storage diseases. Preferably, the internal standard comprises a reference counterpart of each biomarker part of the plurality of biomarkers each indicative for lysosomal storage diseases. Hence, in preferred embodiments the internal standard comprises at least reference versions of at least two biomarkers each indicative for lysosomal storage disease. Optionally, the internal standard comprises 2, 3, 4, 5, 6, 7, 8, 9, 10, or more than 10 reference biomarkers versions each indicative for a distinct lysosomal storage disease, i.e., respectively 2, 3, 4, 5, 6, 7, 8, 9, 10, or more than 10 lysosomal storage diseases.

The internal standard may be any reference biomarker counterpart that allows for calibration of the method, detection of a biomarker, and/or quantification of a biomarker indicative for a lysosomal storage disease. A skilled person appreciates that suitable reference biomarker counterparts are subject of change in function of the detection means that are used for measuring said biomarker. By means of illustration and not limitation, the internal standard may therefore comprise one or more stable isotope labelled reference biomarkers. In such embodiments, the stable isotope labelled reference biomarkers are the counterparts of the biomarkers to be detected.

The term “stable isotope labelled” refers to a molecule that has been the subject of stable isotope labelling. Stable isotope labelling indicates a process where one or more atoms of a molecule is substituted for an atom of the same chemical element having a different isotope. Isotopes of any atom have nearly identical properties, but are characterised by different atomic masses and thereby physical properties due to a different amount of neutrons. Common techniques to detect isotopic differences include (tandem) mass spectrometry (discussed in detail further below), nuclear magnetic resonance (NMR), and autoradiography (e.g. when combined with gel electrophoresis). Tools and techniques to produce stable isotopic labelled molecules for detection methods such as tandem mass spectrometry have been described in the art and are therefore known to a person skilled in the art (e.g. Gevaert, Proteomics, 2008; and Narumi et al., Synth Syst Biotechnol, 2018).

It is evident for a skilled person that “the internal standard” may be a single solution or composition that is added to the sample prior to the detection step of the method, but may equally be a plurality of solutions or compositions that are added to the sample prior to the detection step of the method, e.g., wherein each solution of internal standard comprises a reference counterpart of a biomarker indicative for a lysosomal storage disease. These practicalities and variations relating to the internal standard to be used are considered workshop improvements for a skilled person.

In certain embodiments, the method may further comprise an additional step of mixing of multiple dried blood spot samples derived from different subjects prior to, or during, incubation with the extraction solution. It is envisaged that these embodiments allow for an increased detection (i.e. screening) throughput upon using a suitable detection means as further described in detail below. In further embodiments, the method described herein (steps i) up to and including step iii)) is performed a first time using as input material a collection of blood spot samples derived from different subjects. The number of different subject whereof dried blood spot samples are used is not particularly limited and is appreciated by a skilled person to depend on the sensitivity of the detection means. In such embodiments, if the biomarker is detected by the detection means, the method described herein is performed using in each instance as input material a dried blood spot sample or portion thereof derived from a single subject. It is understood that in these embodiments subjects are screened for the presence of a lysosomal storage disease based on at least two biomarkers by incorporation of an intersubject sample pooling strategy. Optionally, the pooling strategy may be a Carthesian pooling strategy.

The in vitro method described herein and the accompanying kit of parts allow for the detection of at least two different lysosomal storage diseases in a single small blood sample using a single extraction method. Optionally, one of the at least two lysosomal storage diseases is selected from the group of sphingolipidosis, oligosaccharidosis, mucopolysaccharidosis, neuronal ceroid lipofuscinosis, sialic acid disorders, mucolipidosis, cholesteryl ester storage disease, glycogen storage disorders, and cystinosis. Preferably, the at least two lysosomal storage diseases are selected from the group of sphingolipidosis, oligosaccharidosis, mucopolysaccharidosis, neuronal ceroid lipofuscinosis, sialic acid disorders, mucolipidosis, cholesteryl ester storage disease, glycogen storage disorders, and cystinosis.

Optionally, one or both of the at least two lysosomal storage diseases are sphingolipidoses. In further embodiments, one or both of the at least two lysosomal storage diseases are sphingolipidoses preferably selected from the group consisting of: GM2 gangliosidoses. Niemann-Pick disease. Gaucher disease. Fabry disease, metachromatic leukodystrophy, globoid leukodystrophy (Krabbe disease), GM1 gangliosidoses, and multiple sulfatase deficiency. In yet further embodiments, both of the at least two lysosomal storage diseases are sphingolipidoses selected from the group consisting of: acid sphingomyelinase deficiency (i.e. Niemann-Pick disease A/B). Gaucher disease. Fabry disease, and globoid leukodystrophy (Krabbe disease).

In yet further embodiments, one or both of the at least two lysosomal storage diseases are GM2 gangliosidoses selected from the group consisting of: type A (Tay Sachs disease), type O (Sandhoff disease), and type AB (GM2 activator deficiency). In alternative further embodiments, one or both of the at least two lysosomal storage diseases are Niemann-Pick diseases selected from the group consisting of: Niemann-Pick disease A. Niemann-Pick disease B, and Niemann-Pick disease C. In yet alternative further embodiments, one or both of the at least two lysosomal storage diseases are acid sphingomyelinase-deficient Niemann-Pick Disease (ASMD) type A or ASMD type B. In alternative further embodiments, one or both of the at least two lysosomal storage diseases are Gaucher disease types selected from the group consisting of: Gaucher disease type 1. Gaucher disease type 2, and Gaucher disease type 3. In alternative further embodiments, one or both of the at least two lysosomal storage diseases are Fabry disease types selected from the group consisting of: classic type Fabry disease, and late-onset Fabry disease type. In alternative further embodiments, one or both of the at least two lysosomal storage diseases are GM1 gangliosidoses selected from the group consisting of GM1 gangliosidosis type 1, GM1 gangliosidosis type 2, and GM1 gangliosidosis type 3. Optionally, one or both of the at least two lysosomal storage diseases are oligosaccharidosis selected from the group consisting of: alfa mannosidosis, Schindler disease, aspartylglucosaminuria, and fucosidosis.

Optionally, one or both of the at least two lysosomal storage diseases are mucopolysaccharidoses selected from the group consisting of: Hurler syndrome, Scheie syndrome, Hurler-Scheie syndrome, Hunter syndrome, SanFilippo syndrome A, B, C, or D, Morquio syndrome A or B, Maroteaux-Lamy syndrome, and Sly syndrome.

Optionally, one or both of the at least two lysosomal storage diseases are neuronal ceroid lipofuscinoses (CLN) selected from the group consisting of: CLN1, CLN2, CLN3, CLN4, CLN5, CLN6, CLN7, CLN8, CLN9, CLN10, CLN11, CLN12, CLN13, and CLN14.

Optionally, one or both of the at least two lysosomal storage diseases are sialic acid disorders selected from the group consisting of: galactosialidosis, infantile sialic acid storage disease, Salla disease, and sialuria.

Optionally, one or both of the at least two lysosomal storage diseases are mucolipidoses selected from the group consisting of: sialidosis I, sialidosis II (mucolipidosis I), I-cell disease (mucolipidosis II), pseudo-Hurler-Polydystrophy (Mucolipidosis III), and mucolipidosis IV.

Optionally, one or both of the at least two lysosomal storage diseases are glycogen storage disorders selected from the group consisting of: Pompe disease and Danon disease.

Hence, it is envisaged that the method and accompanying kit of parts described herein allow the detection of one or more lysosomal storage diseases selected from the group consisting of: GM2 gangliosidosis type A (Tay Sachs disease), GM2 gangliosidosis type O (Sandhoff disease), GM2 gangliosidosis type AB (GM2 activator deficiency), Niemann-Pick disease A, Niemann-Pick disease B. Niemann-Pick disease C, Gaucher disease type 1, Gaucher disease type 2, Gaucher disease type 3, classic type Fabry disease, late-onset Fabry disease type, metachromatic leukodystrophy, globoid leukodystrophy (Krabbe disease), GM1 gangliosidosis type 1, GM1 gangliosidosis type 2, GM1 gangliosidosis type 3, multiple sulfatase deficiency, alfa mannosidosis, Schindler disease, aspartylglucosaminuria, fucosidosis, Hurler syndrome, Scheie syndrome, Hurler-Scheie syndrome, Hunter syndrome, SanFilippo syndrome A, SanFilippo syndrome B, SanFilippo syndrome C, SanFilippo syndrome D, Morquio syndrome A, Morquio syndrome B, Maroteaux-Lamy syndrome, Sly syndrome, CLN1, CLN2, CLN3, CLN4, CLN5, CLN6, CLN7, CLN8, CLN9, CLN10, CLN11, CLN12, CLN13, and CLN14, galactosialidosis, infantile sialic acid storage disease, Salla disease, sialuria, sialidosis I, sialidosis II (mucolipidosis I), I-cell disease (mucolipidosis II), pseudo-Hurler-Polydystrophy (Mucolipidosis III), and mucolipidosis IV, cholesteryl ester storage disease, Pompe disease, Danon disease, and cystinosis.

Alternatively, the lysosomal storage diseases that may be detected by the method and accompanying kit of parts described herein may be characterised by the type of protein that is defective. Hence, the lysosomal storage diseases may be a lysosomal storage disease that is characterised by a primary deficiency of a lysosomal enzyme, a lysosomal storage disease that is characterised by a missing or aberrant posttranslational modification of a lysosomal enzyme, a lysosomal storage disease that is characterised by a deficiency in a membrane transport protein, a lysosomal storage disease that is characterised by a deficiency in enzyme protecting proteins, a lysosomal storage disease that is characterised by a deficiency in a soluble nonenzymatic protein, or a lysosomal storage disease that is characterised by a deficiency in a transmembrane protein.

In particularly preferred embodiments, the method (or the accompanying kit of parts) described herein is for detection of at least two lysosomal storage diseases, wherein at least one lysosomal storage disease is Gaucher disease and at least one lysosomal storage disease is Fabry disease. In a yet more preferred embodiment, the method (or the accompanying kit of parts) described herein is for detecting a plurality of (i.e. multiple) lysosomal storage diseases, wherein at least one lysosomal disease is Gaucher disease, at least one lysosomal disease is Fabry disease, and at least one lysosomal storage disease is acid sphingomyelinase deficiency.

Optionally, at least one of the plurality of biomarkers used in the method of the present invention that are each indicative for a lysosomal storage disease is a biomarker for a lysosomal storage disease selected from the group of: sphingolipidosis, oligosaccharidosis, mucopolysaccharidosis, neuronal ceroid lipofuscinosis, sialic acid disorders, mucolipidosis, cholesteryl ester storage disease, glycogen storage disorders, and cystinosis. Preferably, at least one of the plurality of biomarkers indicative for a lysosomal storage disease is a biomarker for a lysosomal storage disease selected from the group of: sphingolipidosis, oligosaccharidosis, mucopolysaccharidosis, neuronal ceroid lipofuscinosis, sialic acid disorders, mucolipidosis, cholesteryl ester storage disease, glycogen storage disorders, and cystinosis.

Optionally, at least one of the plurality of biomarkers indicative for a lysosomal storage disease is a biomarker for a sphingolipidosis. In further embodiments, at least one of the plurality of biomarkers is indicative for a lysosomal storage disease selected from the group consisting of: GM2 gangliosidoses, Niemann-Pick disease, Gaucher disease, Fabry disease, metachromatic leukodystrophy, globoid leukodystrophy (Krabbe disease), GM1 gangliosidoses, and multiple sulfatase deficiency. In yet further embodiments, both of the at biomarkers for at least two lysosomal storage diseases are indicative for a sphingolipidosis selected from the group consisting of: acid sphingomyelinase deficiency (i.e. Niemann-Pick disease A/B), Gaucher disease, Fabry disease, and globoid leukodystrophy (Krabbe disease).

In yet further embodiments, at least one of the plurality of biomarkers indicative for a lysosomal storage disease is a biomarker for a GM2 gangliosidosis selected from the group consisting of: type A (Tay Sachs disease), type O (Sandhoff disease), and type AB (GM2 activator deficiency). In alternative further embodiments, at least one of the plurality of biomarkers indicative for a lysosomal storage disease is a biomarker for a Niemann-Pick disease selected from the group consisting of: Niemann-Pick disease A, Niemann-Pick disease B, and Niemann-Pick disease C. In yet alternative further embodiments, at least one of the plurality of biomarkers indicative for a lysosomal storage disease is a biomarker for acid sphingomyelinase-deficient Niemann-Pick Disease (ASMD) type A or ASMD type B. In alternative further embodiments, at least one of the plurality of biomarkers indicative for a lysosomal storage disease is a biomarker for a Gaucher disease type selected from the group consisting of: Gaucher disease type 1, Gaucher disease type 2, and Gaucher disease type 3. In alternative further embodiments, at least one of the plurality of biomarkers indicative for a lysosomal storage disease is a biomarker for a Fabry disease type selected from the group consisting of: classic type Fabry disease, and late-onset Fabry disease type. In alternative further embodiments, at least one of the plurality of biomarkers indicative for a lysosomal storage disease is a biomarker for a GM1 gangliosidosis selected from the group consisting of GM1 gangliosidosis type 1, GM1 gangliosidosis type 2, and GM1 gangliosidosis type 3. Optionally, at least one of the plurality of biomarkers indicative for a lysosomal storage disease is a biomarker for an oligosaccharidosis selected from the group consisting of: alfa mannosidosis, Schindler disease, aspartylglucosaminuria, and fucosidosis.

Optionally, the biomarker is a sphingolipid, or a molecule derived from a sphingolipid. Optionally, the biomarker, or preferably each biomarker, is a lyso-sphingolipid.

In particular embodiments, at least one biomarker is indicative for Gaucher disease and at least one biomarker is indicative for Fabry disease. Preferably, at least one biomarker is indicative for Gaucher disease, at least one biomarker is indicative for Fabry disease, and optionally one biomarker is indicative for acid sphingomyelinase deficiency or Krabbe disease. More preferably, at least one biomarker is indicative for Gaucher disease, at least one biomarker is indicative for Fabry disease, and optionally at most two other biomarkers are indicative for respectively acid sphingomyelinase deficiency and Krabbe disease.

Hence, it is envisaged that the method and accompanying kit of parts described herein allow the detection of one or more biomarkers indicative for lysosomal storage diseases selected from the group consisting of: GM2 gangliosidosis type A (Tay Sachs disease), GM2 gangliosidosis type O (Sandhoff disease), GM2 gangliosidosis type AB (GM2 activator deficiency), Niemann-Pick disease A, Niemann-Pick disease B. Niemann-Pick disease C, Gaucher disease type 1, Gaucher disease type 2, Gaucher disease type 3, classic type Fabry disease, late-onset Fabry disease type, metachromatic leukodystrophy, globoid leukodystrophy (Krabbe disease), GM1 gangliosidosis type 1, GM1 gangliosidosis type 2, GM1 gangliosidosis type 3, multiple sulfatase deficiency, alfa mannosidosis, Schindler disease, aspartylglucosaminuria, fucosidosis, Hurler syndrome, Scheie syndrome, Hurler-Scheie syndrome, Hunter syndrome. SanFilippo syndrome A, SanFilippo syndrome B. SanFilippo syndrome C. SanFilippo syndrome D, Morquio syndrome A. Morquio syndrome B. Maroteaux-Lamy syndrome, Sly syndrome, CLN1, CLN2, CLN3, CLN4, CLN5, CLN6, CLN7, CLN8, CLN9, CLN10, CLN11, CLN12, CLN13, and CLN14, galactosialidosis, infantile sialic acid storage disease, Salla disease, sialuria, sialidosis I, sialidosis II (mucolipidosis I), I-cell disease (mucolipidosis II), pseudo-Hurler-Polydystrophy (Mucolipidosis III), and mucolipidosis IV, cholesteryl ester storage disease, Pompe disease. Danon disease, and cystinosis.

Alternatively, the biomarkers indicative for lysosomal storage diseases that used by the method and accompanying kit of parts described herein may be indicative for a lysosomal storage disease characterised by the type of protein that is defective. Hence, at least one of the plurality of biomarkers may be indicative for a lysosomal storage disease that is characterised by a primary deficiency of a lysosomal enzyme, a lysosomal storage disease that is characterised by a missing or aberrant posttranslational modification of a lysosomal enzyme, a lysosomal storage disease that is characterised by a deficiency in a membrane transport protein, a lysosomal storage disease that is characterised by a deficiency in enzyme protecting proteins, a lysosomal storage disease that is characterised by a deficiency in a soluble nonenzymatic protein, or a lysosomal storage disease that is characterised by a deficiency in a transmembrane protein.

In particularly preferred embodiments, the method (or the accompanying kit of parts) described herein is for detection of at least two lysosomal storage diseases, wherein at least one biomarker is indicative for Gaucher disease and at least one biomarker is indicative for Fabry disease. In further embodiments, the method (or the accompanying kit of parts) described herein is for detecting two biomarkers each indicative for a different lysosomal storage disease, wherein one biomarker is indicative for Gaucher disease and one biomarker is indicative for Fabry disease. In a yet more preferred embodiment, the method (or the accompanying kit of parts) described herein is for detecting multiple biomarkers indicative for lysosomal storage diseases, wherein at least one biomarker is indicative for Gaucher disease, at least one biomarker is indicative for Fabry disease, and at least one biomarker is indicative for acid sphingomyelinase deficiency. In further embodiments, the method (or the accompanying kit of parts) described herein is for detecting three biomarkers each indicative for a different lysosomal storage disease, wherein one biomarker is indicative for Gaucher disease, one biomarker is indicative for Fabry disease, and one biomarker is indicative for acid sphingomyelinase deficiency.

The pathology “Gaucher disease” has been described at numerous instances in the art (e.g., in Stirnemann et al., Int J Mol Sci. 2017) and is therefore well known to a skilled person. Gaucher disease is the most common sphingolipidosis and is caused by a deficiency of the glucocerebrosidase lysosomal enzyme, which results in accumulation of the glucosylceramide substrate in macrophages. Gaucher disease is an autosomal, recessive genetic disease usually caused by mutations in the GBA1 gene (situated on chromosome 1 (1q21): OMIM entry MIM606463). Rare instances of Gaucher disease caused by a deficiency in saposin C have also been described (Vaccaro et al., Hum. Mol. Genet. 2010). Three clinical forms of Gaucher disease have been described in the art, which are each equally envisaged in the context of the present invention: type I (non-neuropathic), type II (acute infantile neuropathic), and type III (chronic neuropathic). Each of these types are linked to groups of specific mutations; Type I: N370S homozygote; Types II and III: L444P. A skilled person appreciates that higher Lyso-Gb1 (interchangeably indicated in the art with the term “GlcSph”) values are associated with more severe disease types. Hence, the present method is capable of stratifying Gaucher patients into Gaucher type I, type II, or type III based on the measured Lyso-Gb1 level. A skilled person appreciates that the abbreviations Lyso-Gb1 and GlcSph, in accordance with the art, both refer to glucosylsphingosine. Hence, each instance of the present application wherein reference is made to the abbreviation “Lyso-Gb1” intends to refer to “glucosylsphingosine”, and said abbreviation may consequently be exchanged for “GlcSph” or “glucosylsphingosine”.

Gaucher disease is characterised by a wide array of symptoms which include without limitation hepatomegaly, splenomegaly, hypersplenism, pancytopenia, anaemia, neutropenia, leukopenia, thrombocytopenia, liver cirrhosis, joint pain, bone pain, osteoporosis, and an abnormal brown skin pigmentation. In addition, neurological symptoms may be present dependent on the type of Gaucher disease: type I: Parkinson disease, impaired cognition; type II: convulsions, hypertonia, intellectual disability, skin abnormalities, apnea; type III: myoclonus, convulsions, dementia, ocular muscle apraxia, and paralysis. It is known that there is considerable overlap between the symptoms of different Gaucher disease types.

In particular embodiments, the obtained Lyso-Gb1 value is compared with a reference range to determine whether a subject has Gaucher Disease, or to determine whether a treatment is effective in a subject diagnosed to have Gaucher Disease. The inventors have determined through extensive experimentation a set of age dependent parametric central 95 percent reference ranges for Lyso-Gb1 detected in a dried blood sample which indicate a healthy status of a subject (i.e., the non-occurrence of Gaucher disease in said subject). For example, a suitable reference range for a healthy subject up to about 4 years old is from 0.282 ng/ml to 6.890 ng/ml; is from 0.235 ng/ml to 5.409 ng/ml for a healthy subject from about 4 years old to about 12 years old; is from 0.237 ng/ml to 6.385 ng/ml for a healthy subject from about 12 years old to about 18 years old; is from 0.272 ng/ml to 7.111 ng/ml for a healthy subject from about 18 years old to about 40 years old; is from 0.319 ng/ml to 6.392 ng/ml for a healthy subject from about 40 years old to about 60 years old; is from 0.317 ng/ml to 4.928 ng/ml for a healthy subject over 60 years old. In embodiments where a Lyso-Gb1 value is obtained that exceeds the boundaries of the appropriate above-mentioned reference range, a diagnosis of Gaucher disease can be made, optionally after conducting further diagnostic practices.

Treatment strategies for Gaucher disease have been described in the art. The present invention therefore encompasses a method of providing information as to the subject's sensitivity to Gaucher disease treatment strategies described in the art, encompassing both enzyme replacement and substrate reduction strategies and hence including but not limited to glucocerebrosidase replacement therapy, glucocerebroside formation inhibition, inhibition of glucosylceramide synthetase, and gene therapy. Thus, the present invention therefore encompasses a method of providing information as to the subject's sensitivity to Gaucher disease treatment strategies such as treatment with (recombinant) glucocerebrosidase such as Alglucerase, Imiglucerase, Velaglucerase, Taliglucerase alfa, Miglustat, and Eliglustat.

The pathology “Fabry disease” (additionally indicated as “Anderson-Fabry disease”) has been described at numerous instances in the art (e.g., in Dinu and Firu, Rom J Morphol Embryol, 2021) and is therefore well known to a skilled person. Fabry disease is an X-linked inherited disorder affecting the glycosphingolipid metabolism caused by mutations in the galactosidase alpha (GLA) gene. The galactosidase alpha gene is responsible for encoding the lysosomal enzyme α-galactosidase (AGAL; OMIM entry MIM301500). The function of AGAL is to metabolize a sphingolipid called globotriaosylceramide (interchangeably indicated in the art by the abbreviations “Gb3” and “GL3”). In Fabry disease, Gb3 is not metabolized and accumulates within lysosomes and other tissues. In accordance with what is described above for Lyso-Gb1 and Gaucher disease, a higher detected level of Lyso-Gb3 may be associated with a predicted worse clinical presentation of Fabry disease (classical versus late-onset phenotype).

Fabry disease is associated with a large number of distinct symptoms including full-body pain, acroparesthesia, gastrointestinal pain, kidney failure, chronic kidney disease, heart muscle hypertrophy optionally leading to restrictive cardiomyopathy, abnormal heart rhythms including complete heart block and ventricular tachycardia, regurgitation, stenosis, angiokeratomas, anhidrosis, hyperhidrosis, neuropathy, cornea verticillata, conjunctival and retinal vascular abnormalities, fatigue, tinnitus, vertigo, nausea, inability to gain weight, chemical imbalances, and diarrhoea.

In particular embodiments, the obtained Lyso-Gb3 value is compared with a reference range to determine whether a subject has Fabry Disease, or to determine whether a treatment is effective in a subject diagnosed to have Fabry Disease. The inventors have determined through extensive experimentation a set of age dependent parametric central 95 percent reference ranges for Lyso-Gb3 detected in a dried blood sample which indicate a healthy status of a subject (i.e., the non-occurrence of Fabry disease in said subject). For example, a suitable reference range for a healthy subject up to about 4 years old is from 0.0441 ng/ml to 1.952 ng/ml; is from 0.223 ng/ml to 2.326 ng/ml for a healthy subject from about 4 years old to about 12 years old; is from 0.138 ng/ml to 2.014 ng/ml for a healthy subject from about 12 years old to about 18 years old; is from 0.145 ng/ml to 1.971 ng/ml for a healthy subject from about 18 years old to about 40 years old; is from 0.085 ng/ml to 2.014 ng/ml for a healthy subject from about 40 years old to about 60 years old; is from 0.118 ng/ml to 1.774 ng/ml for a healthy subject over 60 years old. Preferably, a suitable reference range for a female healthy subject up to about 4 years old is from 0.0441 ng/ml to 1.518 ng/ml and/or for a male healthy subject up to about 4 years old is from 0.057 ng/ml to 1.952 ng/ml. Preferably, a suitable reference range for a female healthy subject from about 4 years old to about 12 years old is from 0.262 ng/ml to 2.217 ng/ml and/or for a male healthy subject from about 4 years old to about 12 years old is from 0.223 ng/ml to 2.326 ng/ml. In embodiments where a Lyso-Gb3 value is obtained that exceeds the boundaries of the appropriate above-mentioned reference range, a diagnosis of Fabry disease can be made, optionally after conducting further diagnostic practices.

Treatment strategies for Fabry disease have been described in the art. The present invention therefore encompasses a method of providing information as to the subject's sensitivity to Fabry disease treatment strategies described in the art, encompassing both enzyme replacement and substrate reduction strategies and hence including but not limited to enzyme replacement therapy, gene therapy, and pharmacological chaperone therapy. Thus, the present invention therefore encompasses a method of providing information as to the subject's sensitivity to Fabry disease treatment strategies such as treatment with (recombinant) α-galactosidase such as agalsidase alfa, agalsidase beta, migalastat, pegunigalsidase alfa, and gene therapy.

“Acid sphingomyelinase deficiency” or “ASMD” as referred to herein and historically interchangeably referred to in the art as Niemann-Pick type A or B indicate diseases caused by mutations in the sphingomyelin phosphodiesterase 1 (SMPD1) gene, which in healthy subjects is responsible for the production of acid sphingomyelinase lysosomal enzyme. A deficiency or absence of acid sphingomyelinase results in accumulation of sphingomyelin. Acid sphingomyelinase deficiency has been described in detail at numerous instances throughout the art (e.g., in Cox et al., JIMP Rep, 2018). Symptoms of Niemann-Pick type A and B differ. Common symptoms in Niemann-Pick type A that are typically observed during infancy include icterus, enlarged liver, failure to thrive, progressive deterioration of the nervous system, and brain damage. Life expectancy of Niemann-Pick type A is generally at most 18 months. Symptoms of Niemann-Pick type B that are typically observed during the childhood of a subject include hepatosplenomegaly, growth retardation, and lung disorders, abnormal cholesterol level, abnormal lipid levels, and blood clotting without brain damage.

The in vitro method described herein may be a method for detecting multiple biomarkers indicative for any of the lysosomal storage diseases described herein from a dried blood spot sample. Preferably, one or more of the plurality of biomarkers are selected from the group consisting of: glucosylceramide (Gb1), glucosylsphingosine (Lyso-Gb1), Lyso-Gb1 analogs, methylated Gb1 isoforms, non-methylated Gb1 isoforms globotriaosylceramide (Gb3), globotriaosylsphingosine (Lyso-Gb3), Lyso-Gb3 analogs, methylated Gb3 isoforms, non-methylated Gb3 isoforms, glucosylceramide, lysosphingomyelin-509, lysosphingomyelin (Lyso-SPM), chitotriosidase (ChT), pulmonary and activation-regulated chemokine (CCL18/PARC), galactosylceramide, galactosylsphingosine/psychosine, and glycosaminoglycans/GAGS, dermatan sulfate, heparan sulfate, keratan sulfate, chondroitin-6-sulfate, and chondroitin-4,6-sulfate.

More preferably, each of the plurality of biomarkers are selected from the group consisting of: glucosylceramide (Gb1), glucosylsphingosine (Lyso-Gb1), globotriaosylceramide (Gb3), globotriaosylsphingosine (Lyso-Gb3), LysoGb3 analogs, methylated Gb3 isoforms, non-methylated Gb3 isoforms, glucosylceramide, lysosphingomyelin-509, lysosphingomyelin (Lyso-SPM), chitotriosidase (ChT), pulmonary and activation-regulated chemokine (CCL18/PARC), macrophage inflammatory protein 1-alpha (MIP-la), galactosylceramide, galactosylsphingosine/psychosine, glycosaminoglycans/GAGs, dermatan sulfate, heparan sulfate, keratan sulfate, chondroitin-6-sulfate, and chondroitin-4,6-sulfate.

Optionally, the plurality of biomarkers comprises at least two glycosphingolipids.

Yet more preferably, one or more of the plurality of biomarkers are selected from the group consisting of: glucosylsphingosine (Lyso-Gb1), globotriaosylsphingosine (Lyso-Gb3), lysosphingomyelin-509, and lysosphingomyelin (Lyso-SPM). Even more preferably, each of the plurality of biomarkers are selected from the group consisting of: glucosylsphingosine (Lyso-Gb1), globotriaosylsphingosine (Lyso-Gb3), lysosphingomyelin-509, and lysosphingomyelin (Lyso-SPM).

In highly preferable embodiments, at least one of the biomarkers is glucosylsphingosine (Lyso-Gb1) or globotriaosylsphingosine (Lyso-Gb3). Most preferably, a first biomarker is glucosylsphingosine (Lyso-Gb1) and a second biomarker is globotriaosylsphingosine (Lyso-Gb3). Optionally, a third biomarker is lysosphingomyelin (Lyso-SPM). In such embodiments, glucosylsphingosine (Lyso-Gb1) is indicative for Gaucher disease, globotriaosylsphingosine (Lyso-Gb3) is indicative for Fabry disease, and the optional lysosphingomyelin (Lyso-SPM) biomarker is indicative for acid sphingomyelinase deficiency.

The biomarker “glucosylsphingosine”, interchangeably indicated herein by the commonly accepted abbreviation “Lyso-Gb1”, is a known biomarker for Gaucher disease and has been described in the art (e.g., in Hurvitz et al., Glucosylsphingosine (lyso-Gb1) as a biomarker for monitoring treated and untreated children with Gaucher disease. Int J Mol Sci. 2019). Further synonyms of glucosylsphingosine include without limitation glucosyl-C18-sphingosine. (3R,4S,5S,6R)-2-[(E)-2-amino-3-hydroxyoctadec-4-enoxy]-6-(hydroxymethyl) oxane-3,4,5-triol, glucopsychosine, glucosylsphingosine, glucosyl psychosine, glucosyl sphingosine, and sphingosyl beta-glucoside. Glucosylsphingosine (lyso-Gb1) is a downstream metabolic product of glucosylceramide (i.e, a deacylated form of glucosylceramide). Glucosylsphingosine is characterised by PubChem CID 22833534 and molecular formula C₂₄H₄₇NO₇.

The biomarker “globotriaosylsphingosine”, interchangeably indicated herein by the commonly accepted abbreviation “Lyso-Gb3”, is a known biomarker for Fabry disease and has been described in the art (e.g., in Aerts et al., Elevated globotriaosylsphingosine is a hallmark of Fabry disease. Proc Natl Acad Sci USA. 2008). Further synonyms of globotriaosylsphingosine include without limitation alpha-D-galactopyranosyl-(1.4)-beta-D-galactopyranosyl-(1.4)-beta-D-glucopyranosyl-(1.1)-(2S,3R,4E)-2-amino-octadec-4-ene-1,3-diol. Gb3 lysosphingolipid, globotriaosyl lysosphingolipid. Lyso-GL-3. Lyso-globotriaosylsphingosine. (2R,3R,4S,5R,6R)-2-[(2R,3R,4R,5R,6S)-6-[(2R,3S,4R,5R,6R)-6-[(E,2S,3R)-2-amino-3-hydroxyoctadec-4-enoxy]-4,5-dihydroxy-2-(hydroxymethyl) oxan-3-yl]oxy-4,5-dihydroxy-2-(hydroxymethyl) oxan-3-yl]oxy-6-(hydroxymethyl) oxane-3,4,5-triol, and (R—(R*,S*-(E)))-2-Amino-3-hydroxy-4-octadecenyl O-alpha-D-galactopyranosyl-(1-4)-O-beta-D-galactopyranosyl-(1-4)-beta-D-glucopyranoside. Globotriaosylsphingosine is characterised by PubChem CID 6449939 and molecular formula C₃₆H₆₇NO₁₇.

The biomarker “lysosphingomyelin”, interchangeably indicated herein by the commonly accepted abbreviations “Lyso-SM” and “Lyso-SPM”, is a known biomarker for acid sphingomyelinase deficiency and has been described in the art (e.g., in Sheck Breilyn et al., Mol Genet Metab Rep. 2021). Further synonyms of lysosphingomyelin include sphingosylphosphorylcholine, sphing-4-enine-1-phosphocholine, sphingosylphosphocholine acid, and D-erythro-sphingosylphosphorylcholine. Lysosphingomyelin is characterised by PubChem CID 5280613 and molecular formula C₂₃H₄₉N₂O₅P⁺.

In view of the above, a highly preferred internal standard as described above comprises, consists essentially of, or consists of a stable isotope labeled reference Lyso-Gb1 biomarker and a stable isotope labeled reference Lyso-Gb3 biomarker. A further highly preferred internal standard as described above comprises, consists essentially of, or consists of a stable isotope labeled reference Lyso-Gb1 biomarker, a stable isotope labeled reference Lyso-Gb3 biomarker, and a stable isotope labeled reference Lyso-SPM biomarker. By means of illustration, a suitable stable isotope labeled reference Lyso-Gb1 biomarker is ¹³C₆-Lyso-Gb1 (such as commercially available from e.g. Matreya LLC Lipids and biochemical). By means of illustration, a suitable stable isotope labeled reference Lyso-Gb3 biomarker is ¹³C₆-Lyso-Gb3 (such as commercially available from e.g. Gelb Chem. LLC).

The detected Lyso-Gb1 and/or Lyso-Gb3 value may be compared with one or more reference values described above wherein said reference values are indicative for a healthy status of the subject from which the dried blood spot sample is obtained. “A healthy status” in this context indicates the non-occurrence of respectively Gaucher disease and Fabry disease.

Preferably, the method described herein is characterized by i) an extraction step of the dried blood spot sample or portion thereof for about 20 minutes at 37° C. under agitation in an extraction solution comprising about 50% DMSO and about 50% of methanol: ii) a centrifugation step comprising centrifugation for at about 10 minutes at about 5250 g at about 20° C. of the sample obtained in step i) and collecting the supernatant; and iii) a detection step comprising detection of at least Lyso-Gb1 and Lyso-Gb3 in the supernatant of step ii) indicative for respectively Gaucher disease and Fabry disease. More preferably, the method described herein is characterized by i) an extraction of the dried blood spot sample or portion thereof for about 20 minutes at 37° C. under agitation in an extraction solution comprising about 50% DMSO and about 50% of methanol: ii) a centrifugation step comprising centrifugation for at about 10 minutes at about 5250 g at about 20° C. of the sample obtained in step i) and collecting the supernatant; and iii) a detection step comprising detection of at least Lyso-Gb1, Lyso-Gb3, and Lyso-SPM in the supernatant of step ii) indicative for respectively Gaucher disease, Fabry disease, and acid sphingomyelinase deficiency.

The method and kits described herein are at least suitable to predict an outcome of Gaucher disease treatment and/or Fabry disease treatment on a subject. The term “outcome” generally refers to the evaluation undertaken to assess the results or consequences of management and procedures (i.e., the interventions) used in combatting a disease in order to determine the efficacy, effectiveness, safety, practicability, etc., of these interventions, e.g., in individual cases or series. The phrase “predicting outcome” as used herein refers to a process of assessing the consequences of treating a subject for a lysosomal storage disease which the subject is determined to have based on the detection of one of the biomarkers of the present method above a certain threshold level, and predicting whether said individual is likely to respond or not to the treatment. The method of the invention thus at least provides a prediction of how the clinical image of a subject would change and whether there is chance of one or more clinical symptoms characterising the subject.

The terms “treat” or “treatment” encompass the therapeutic treatment of an already developed disease or condition, such as the therapy of both an already developed clinical image indicative for a lysosomal storage disease or an anticipated clinical image indicative for a lysosomal storage disease (i.e., a clinical image that is expected to occur in a future point in time). Beneficial or desired clinical results may include, without limitation, alleviation of one or more symptoms or one or more biological markers, diminishment of extent of disease, stabilized (i.e. not worsening) state of disease, delay or slowing of disease progression, amelioration or palliation of the disease state, and the like. “Treatment” can also mean prolonging survival as compared to expected survival if not receiving treatment.

The terms “predicting”, “prediction” or “predictive” as used herein refers to an advance declaration, indication or foretelling of a response or reaction to a therapy in a subject, preferably wherein said subject has not (yet) been treated with the therapy. For example, a prediction of sensitivity (or responsiveness or susceptibility) to enzyme replacement therapy in a subject may indicate that the subject will respond or react to the treatment, for example within a certain time period, e.g., so that the subject will have a clinical benefit (e.g., will display less clinical symptoms or a diminished disease progression) from the treatment. A prediction of insensitivity (or unresponsiveness or insusceptibility or resistance) to for example enzyme replacement treatment in a subject may indicate that the subject will minimally or not respond or react to the treatment, for example within a certain time period, e.g., so that the subject will have no clinical benefit (e.g., will not display a therapeutically meaningful reduction in unwanted build-up of certain biomolecules in lysosomes) from the treatment.

In addition to the other merits and advantages described throughout the present disclosure, the methods and accompanying kits such as those described herein allow for predicting the onset of clinical (undesirable) manifestations (i.e. symptoms) of lysosomal storage diseases, such as sphingolipidosis, preferably Gaucher disease, Fabry disease, and optionally acid sphingomyelinase deficiency (ASMD) and Krabbe disease. Optionally, the methods and accompanying kits as described herein allow predicting the age on which clinical manifestations will become apparent in a subject if said subject is considered to have a lysosomal storage disease. For example, a late onset variant of Fabry disease may already be diagnosed by the method and predicted to become clinical relevant at a certain age upon new-born screening. In certain embodiments, the method allows to predict whether symptoms will substantially become apparent in a juvenile or adult age of the subject. Alternatively, the method may allow to predict whether clinical symptoms will become apparent within a time span of 1, 2, 5, 10, 15, 20, or more than 20 years in the future lifespan of the tested subject.

The terms “sensitivity”, “responsiveness” or “susceptibility” may be used interchangeably herein and refer to the quality that predisposes a subject having a lysosomal storage disease to be sensitive or reactive to a certain treatment. A subject is “sensitive”, “responsive” or “susceptible” (which terms may be used interchangeably) to a certain treatment if the subject will have a clinical benefit from the treatment.

The terms “insensitivity”, “unresponsiveness”, “insusceptibility” or “resistance” may be used interchangeably herein and refer to the quality that predisposes a subject having a lysosomal storage disease to a minimal (e.g. clinically insignificant) or no response to treatment with a treatment directed to a certain lysosomal storage disease. A subject is considered “insensitive”, “unresponsive”, “unsusceptible” or “resistant” (used interchangeably in the art) to said treatment if the subject will have no clinical benefit from the treatment.

Similarly, the method subject of the invention and the accompanying kits allow for predicting a certain likeliness of response or reaction of a subject to a lysosomal storage disease treatment based on the detected value of the plurality of biomarkers included in the method. “Determining the likeliness of” and “predicting the likeliness of” as used herein refers to an advance declaration, indication or foretelling of a response or reaction to a therapy in a subject, or a probability of a response or reaction to a therapy in a subject, preferably wherein said subject has not (yet) been treated with the therapy

The method subject of the invention may be conducted more than once for a given blood spot sample or portion thereof. Thus, the method may be performed on different portions of the dried blood spot sample to provide a certain desired robustness, e.g., by considering the repetitions of the method as technical repeats. Alternatively or additionally, the detection step may be repeated a number of times, which could also be considered technical repeats. Additionally, the method may be conducted on blood spot samples (or portions thereof) obtained from a subject at different points in time. For example, the method may be conducted a first time on a dried blood spot sample obtained from the subject prior to starting a lysosomal storage disease treatment, and a second time on a dried blood spot sample obtained from the subject during or after the start of treatment for a lysosomal storage disease. Hence, in such embodiments the method can equally act as a treatment monitoring method. Complementary, the method can act as a method to monitor efficacy of novel lysosomal storage disease treatments whereof efficacy has not been established in the art.

The inventors have found that the method described herein is compatible with different detection means. Suitable detection means for one or more markers that is to be detected include sequencing assays, microarrays (e.g., proteome arrays), antibody-binding assays. Enzyme-Linked Immunosorbent Assays (ELISAs), flow cytometry, protein assays, western blots, nephelometry, turbidimetry, chromatography, mass spectrometry, and immunoassays. Illustrative examples of immunoassays include radioimmunoassay's (RIA), immunofluorescence, immunochemiluminescence, immunoelectrochemiluminescence, competitive immunoassays, and immunoprecipitation. Thus, the invention encompasses both detection steps, means, and methods that detect and optionally quantify the biomarkers by direct detection of said biomarkers, but equally encompasses detection steps, means, and methods that detect and optionally quantify biomarkers by detecting binding of said biomarkers to a second moiety, such as but not limited to binding agents. Non-limiting examples of suitable binding agents include antibodies, antibody fragments, antibody-like protein scaffolds, aptamers, spiegelmers (L-aptamers), photo aptamers, proteins, peptides, peptidomimetics, nucleic acids such as oligonucleotides (for example hybridization probes, amplification primers, sequencing primers, and primer pairs), small molecules, and any combination thereof.

In certain embodiments, the method is a method for discriminating between the different lysosomal storage diseases, more particularly between Gaucher disease and Fabry disease, i.e., a method of determining whether the patient is suffering from Gaucher disease or Fabry disease. In further embodiments, the method is a method for discriminating between Gaucher disease, Fabry disease, and acid sphingomyelinase deficiency i.e., a method of determining whether the patient is suffering from Gaucher disease, Fabry disease, and acid sphingomyelinase deficiency.

Preferably, the detection step of the method comprises detection of at least one biomarker by mass spectrometry. More preferably, the detection step of the method comprises detection of at least one biomarker by tandem mass spectrometry. Yet more preferably, the detection step of the method comprises detection of at least two biomarkers by tandem mass spectrometry. Yet more preferably, the detection step of the method comprises detection of at least 3, at least 4, at least 5, at least 6, at least 7, at least 8, at least 9, at least 10, or even more than 10 biomarkers by tandem mass spectrometry. Most preferably, the detection step of the method comprises detection of all biomarkers detected in the method.

The term “mass spectrometry” as used herein broadly refers to techniques that are capable of measuring mass-to-charge ratios (commonly indicated in the art by “m/z” or “m/Q”) of ions. Said techniques are well-known to a person skilled in the art. Generally, mass spectrometers comprise three main components: an ion source, a mass analyser, and a detector. Generally, in a first step ions are generated of the analyte which may optionally involve fragmentation. Subsequently, the ions are separated from each other based on mass-to-charge ratio. Finally, detection occurs by a detector. Non-limiting ionization techniques include electrospray ionization (ESI), Atmospheric Pressure Chemical Ionization, Atmospheric Pressure Photoionization, matrix-assisted laser desorption/ionization (MALDI), Gas-Phase Protonation, Ambient Desorption Ionization, Desorption Electrospray Ionization (DESI), Direct Analysis in Real Time (DART), and fast atom bombardment (FAB) (e.g. reviewed in Awad et al. Appl Spectrosc Rev, 2014). Non-limiting examples of mass selectors include Time-of-Flight (TOF) mass filters, quadrupole mass filters, ion trap mass filters, Fourier-transform ion cyclotron resonance mass selectors, such as orbitrap mass filters. Non-limiting examples of ion detectors include electron multipliers, Faraday cups, photomultiplier conversion dynode, and array detectors.

Examples of mass spectrometry methods suitable for use in the method subject of the invention include fast atom bombardment mass spectrometry (FAB-MS), liquid chromatography mass spectrometry (LC-MS), liquid chromatography tandem mass spectrometry (LC-MS/MS), matrix-assisted laser desorption/ionization mass spectrometry (MALDI-MS), matrix-assisted laser desorption/ionization tandem mass spectrometry (MALDI-MS/MS). Preferably, the detection means used in the detection step of the method subject of the invention is liquid chromatography tandem mass spectrometry detection means.

As implied above, the detection step, which may include tandem mass spectrometry, may include one or more chromatography steps. The term “chromatography” encompasses methods for separating substances, such as chemical or biological substances, e.g., markers, such as preferably peptides, polypeptides, or proteins, referred to as such and vastly available in the art. In a preferred interpretation according to the invention, chromatography refers to a process in which a mixture of substances (analytes) carried by a moving stream of liquid or gas, preferably liquid in the context of the present invention (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is widely applicable for the separation of chemical compounds of biological origin. Exemplary types of chromatography include, without limitation, high-performance or high-pressure liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immunoaffinity, and immobilised metal affinity chromatography.

Preferably, the chromatographic method (or chromatographic step) is columnar (i.e., wherein the stationary phase is deposited or packed in a column), preferably liquid chromatography, and yet more preferably high-performance or high-pressure liquid chromatography (HPLC). A skilled person is familiar with HPLC (e.g., Bidlingmeyer, Wiley & Sons Inc., 1993). A non-limiting exemplary HPLC gradient corresponds to a gradient elution comprising as solvent A about 0.1% formic acid in water and as solvent B 0.1% formic acid in 20% methanol and 80% acetonitrile while maintaining a flow rate of about 0.50 ml/min. An exemplary suitable gradient is set at 75% A-25% B at the start of the chromatography process which is gradually changed to 0% A-100% B by 2.5 min after starting the process.

Further techniques for separating, detecting and/or quantifying biomarkers may be used, optionally in conjunction with any of the above described methods. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), and free flow electrophoresis (FFE).

A particularly preferred detection means (i.e., detection method, detection step) is liquid chromatography tandem mass spectrometry (LC-MS/MS). As is evident from the above. LC-MS/MS is a coupled liquid chromatography-mass spectrometry system wherein the liquid sample (i.e, the collected supernatant of the sample obtained in step i) of the method described herein is analysed by mass spectrometry after separation of the sample constituents by chromatography. Typically, the tandem mass spectrometry aspect of the method as such comprises an initial separation of ionized molecules (i.e, the constituents of the sample), preferably by electrospray ionization, in a first spectrometer (i.e., MS1) according to their mass-to-charge ratio. Particular ions characterised by a predefined m/z ratio are then selected for further fragmentation (i.e., fragment ions). Said fragment ions are subsequently introduced into a second spectrometer (i.e., MS2) which performs a further selection according to m/z ratio of the fragments, after which detection occurs.

In certain embodiments, an extraction solution volume of from about 50 to about 250 μl is used in step i) of the method to incubate three punches (each having a size of about ⅛ inches or about 0.3175 cm) of the dried blood sample and of from about 0.5 to about 20 μl of the supernatant obtained in step i) of the method is injected into the LC-MS/MS system. Preferably, an extraction solution of from about 100 to about 200 μl is used in step i) of the method to incubate three punches (each having a size of about ⅛ inches or about 0.3175 cm) of the dried blood spot sample and of from about 0.5 μl to 10 μl of the supernatant obtained in step i) of the method is injected into the LC-MS/MS system. More preferably, an extraction solution of from about 125 to about 175 μl is used in step i) of the method to incubate three punches (each having a size of about ⅛ inches or about 0.3175 cm) of the dried blood spot sample and of from about 0.5 μl to about 5 μl of the supernatant obtained in step i) of the method is injected into the LC-MS/MS system. Most preferably, an extraction solution of about 150 μl is used in step i) of the method to incubate three punches (each having a size of about ⅛ inches or about 0.3175 cm) of the dried blood spot sample and of from about 2 μl of the supernatant obtained in step i) of the method is injected into the LC-MS/MS system.

Optionally, the sample may be further separated prior to introduction into the MS/MS system. By means of example, such a separation may occur by means of a C18 column. “C18 column” is to be interpreted in accordance to the generally accepted meaning within the field of proteomics and mass spectrometry and therefore broadly refers to an octadecyl carbon chain bonded silica column.

Optionally, the mass spectrometry method that may be used as detection means is a targeted tandem mass spectrometry method. Targeted mass spectrometry methods are typically performed on triple quadrupole (i.e., QQQ) mass spectrometers wherein the first quadrupole (Q1) acts as a filter to select predicted precursor molecules, the second quadrupole (Q2) is used as a collision cell to fragment said precursor (i.e. parent) molecules, and the third quadrupole (Q3) detects a predefined fragment m/z (i.e, the daughter molecules). Alternatively targeted tandem mass spectrometry approaches have been described in the art and include quadrupole-orbitrap approaches (Vidova and Spacil, Anal Chim Acta, 2017). Thus, in contrast to discovery (or alternatively “shotgun”) tandem mass spectrometry approaches, targeted tandem mass spectrometry methods rely on a priori obtained knowledge about one or more so-called “transitions” of an analyte of interest. The term “transitions” is well known in the field of proteomics and refers generally to the combination of precursor and fragment m/z values of a molecule of interest (Doer, Nat Methods, 2013). The precise targeted tandem mass spectrometry method is not particularly limiting and may therefore be selected reaction monitoring (SRM), multiple reaction monitoring (MRM), or parallel reaction monitoring (PRM).

As described above, certain embodiments of the present invention comprise a method wherein the level of Lyso-Gb1 and/or Lyso-Gb3 are measured by means of a targeted tandem mass spectrometry method. In such embodiments, preferably the Lyso-Gb1 precursor is measured at an m/z value of from about 460 to about 465, preferably measured at an m/z value of from 461 to about 464, more preferably measured at an m/z value of from about 461.5 to about 463, yet more preferably measured at an m/z value of from about 462 to about 462.5, yet even more preferably at an m/z value of about 462.3, most preferably at an m/z value of about 462.29. In further preferred embodiments, the Lyso-Gb1 precursor is measured at an m/z value of 462.294. The Lyso-Gb3 precursor is measured at an m/z value of about 786.2 to about 786.8, preferably at an m/z value of about 786.2 to 786.5, more preferably at an m/z value of about 786.2 to about 786.4, most preferably at an m/z value of 786.3 m/z or at 786.4 m/z, more specifically 786.392 m/z.

In certain embodiments, the targeted tandem mass spectrometry assay comprises measurement of a parent Lyso-Gb1 ion and a parent Lyso-Gb3 ion, preferably wherein the targeted tandem mass spectrometry assay comprises measurement of a parent Lyso-Gb1 ion at 462.2 to 462.3 m/z and a parent Lyso-Gb3 ion at 786.2 to 786.5 m/z.

In particular embodiments, the targeted tandem mass spectrometry assay comprises measurement of a daughter Lyso-Gb1 ion at 282.0 m/z to 282.5 m/z and/or a daughter Lyso-Gb3 ion at 282.0 m/z to 282.5 m/z. In particular embodiments, the targeted tandem mass spectrometry assay comprises measurement of a daughter Lyso-Gb1 ion at 282.3 m/z and a daughter Lyso-Gb3 ion at 282.3 m/z.

In preferred embodiments, the targeted tandem mass spectrometry assay comprises measurement of a parent Lyso-Gb1 ion at 462.2 to 462.3 m/z and a daughter Lyso-Gb1 ion at 282.0 m/z to 282.5 m/z. In alternative preferred embodiments, the targeted tandem mass spectrometry assay comprises measurement of a parent Lyso-Gb1 ion at 462.294 m/z, and a daughter Lyso-Gb1 ion at 282.3 m/z.

In alternative preferred embodiments, the targeted tandem mass spectrometry assay comprises measurement of a parent Lyso-Gb3 ion at 786.2 to 786.8 m/z and a daughter Lyso-Gb3 ion at 282.0 m/z to 282.5 m/z. In more particular embodiments, the targeted tandem mass spectrometry assay comprises measurement of a parent Lyso-Gb3 ion at 786.392 m/z and a daughter Lyso-Gb3 ion at 282.3 m/z.

In certain embodiments wherein a prediction about the occurrence of Gaucher disease is made based on a dried blood spot sample, a detection of a Lyso-Gb1 level of 1.5 ng/ml or more, preferably 5 ng/ml or more in a subject is indicative for Gaucher disease. In certain embodiments wherein a prediction about the occurrence of Fabry disease is made based on a dried blood spot sample, a detection of a Lyso-Gb3 level of 1 ng/ml or more, preferably 2 ng/ml or more in a subject is indicative for Fabry disease. Optionally, the method described herein comprises a step of conducting one or more enzymatic assays on the dried blood spot sample or portion thereof, or on the supernatant obtained by the centrifugation step of the method. Preferably, the enzymatic assay is directed to determining the enzymatic activity of an enzyme whereof a reduced activity or lack of activity is related to a lysosomal storage disease. More preferably, the enzymatic assays are directed to determining the enzymatic activity of an enzyme whereof a reduced activity or lack of activity is related to Gaucher disease, Fabry disease, and/or acid sphingomyelinase deficiency. By means of illustration and not limitation, suitable enzymatic assays include assays that determine the activity of one or more enzymes selected from the group consisting of glucocerebrosidase (Gaucher disease), alfa-galactosidase (Fabry disease), and acid sphingomyelinase (acid sphingomyelinase deficiency). Enzymatic assays for determining the activity of glucocerebrosidase, alfa-galactosidase, and acid sphingomyelinase have been described in the art and are therefore known to a skilled person (e.g. Ysselstein et al., Mov Disord, 2021: Mayes et al., Clin Chim Acta, 1981; and Chuang et al., Mol Genet Metab Rep, 2015). Alternatively, the present method comprises a step of conducting one or more enzymatic assays on the dried blood spot sample or portion thereof, or on the supernatant obtained in the centrifugation step of the method on enzymes having a substrate selected from the group consisting of: GM1 gangliosides or non-sialylated derivates, sphingolipids, galactosylceramide, psychosine, sulfatides, globotriaosylceramide, glucocerebroside, sphingomyelin, unesterified cholesterol, GM2 gangliosides, mucopolysaccharides, partially degraded oligosaccharides, glycosphingolipids, glycoproteins, oligosaccharides, aspartylglycosamine, N-acetylglucosamine, heparan, dermatan sulphates, heparan sulphates, keratan sulphate, chondroitin-6-sulphate, chondroitin-4-sulphate, chondroitin-4,6-sulphate, sialyloligosaccharides, sialic acid, cholesteryl esters, triglycerides, glycogen, and cystine.

Optionally, the method may further comprise, in addition to the dried blood spot sample analysis, a step of determining enzymatic activity in the blood of the subject from who the dried blood spot sample was obtained. For example, upon detection of a certain level of Lyso-Gb1 in the dried blood spot sample by the method described herein, the enzymatic activity of glucocerebrosidase may be determined in lymphocytes of said subject. A skilled person appreciates that the term “lymphocytes” is to be interpreted as broadly accepted in the technical field of immunology and therefore relates to a type of white blood cells.

Optionally, the method may further comprise, in addition to the dried blood spot sample analysis, genetic analysis of one or more genes known to be causative for a lysosomal storage disease. Thus, the method may comprises an additional step of genetic analysis of one or more genes selected from the group consisting of: GLB1, GALC, ASA, GLA, GBA, SMPD1, NPC1, NPC2, HEXA, HEXB, GM2A. SUMF1, MAN2B1, NAGA, AGA, FUCA1, IDUA, SGSH, NAGLU, HGSNAT, GNS, GALNS, GLB1, ARSB, GUSB, CLN1, CLN2, CLN3, CLN4, CLN5, CLN6, CLN7, CLN8, CLN9, CLN10, CLN11, CLN12, CLN13, CLN14, CTSA, SLC17A5, GNE, NEU1, GNPTAB, MCOLN1, LIPA, GAA, LAMP2, and CTNS. Numerous sequencing methods have been described in the art (e.g., in Goodwin et al., Nat Rev Genet. 2016). The particulars of the genetic analysis are not particularly limited in the context of the present invention and are preferably sequencing analyses. Numerous sequencing techniques are known and widely used in the art including the Sanger method and Gilbert chemical method. Alternative sequencing methods such as pyrosequencing are capable of monitoring DNA synthesis in real time using a luminometric detection system. Pyrosequencing has been shown to be particularly effective in analysing genetic polymorphisms such as single-nucleotide polymorphisms and can also be used in the present invention (Nordstrom et al., Biotechnol Appl Biochem. 2000). Moreover, the term “sequencing analyses” is additionally envisaged to encompass high-throughput sequencing methods such as single-molecule real-time sequencing, ion semiconductor, pyrosequencing, sequencing by synthesis, combinatorial probe anchor synthesis, sequencing by ligation (SOLID), nanopore sequencing, genapsys sequencing, and chain termination sequencing.

As is evident for any skilled person and as indicated above, the method of the invention described herein may incorporate or rely on use of computer-assisted detection and/or analysis means such as software or computer-controlled sensors. The present invention therefore further relates to a computer system comprising a processor, and optionally a memory coupled to said processor and encoding one or more software programs, wherein said one or more software programs instruct the processor to carry out the method subject of the present disclosure. Thus, in any of the embodiments described herein, the method may be a computer-implemented method. In embodiments where the method is a computer-implemented method, the method includes obtaining by a computing device the levels of the plurality of biomarkers present in the dried blood spot sample and, and optionally storing, by the computing device, the probabilistic assessment (i.e, the prediction) of a lysosomal storage disease status based on the measured levels of the plurality of biomarkers, such as comparing said measured levels with reference ranges. For example, in the context of Gaucher disease and Fabry disease, the computer implemented method or the computing device may compare the obtained Lyso-Gb1 value and/or Lyso-Gb3 value with the reference values described above for obtaining a prediction on whether the subject said values are obtained from is considered to have respectively Gaucher disease or Fabry disease. The computing device may obtain the plurality of measured biomarker levels in an automated manner (i.e. without any user intervention), in a semi-automatic manner (e.g. batch input of a group of biomarker levels), or by manual user input of (each of) the measured biomarker levels.

In such embodiments, the computer software typically includes a computer readable medium having computer-executable instructions for performing the logic steps of the method of the invention. Non-limiting examples of a suitable computer readable medium include floppy disks. CD-ROM/DVD/DVD-ROM, a hard-disk drives, flash memory. ROM/RAM, and magnetic tapes. The computer executable instructions may be written in any suitable computer language or combination of several languages. Basic computational biology methods have been described in the art and are therefore known to a skilled person (e.g., Gauthier et al., Brief Bioinform, 2019). Optionally, the computer readable medium may contain information on reference values for Gaucher disease and/or Fabry disease. For example, the computer readable medium may contain the information that the reference Lyso-Gb1 range for a healthy subject up to about 4 years old is from 0.282 ng/ml to 6.890 ng/ml; is from 0.235 ng/ml to 5.409 ng/ml for a healthy subject from about 4 years old to about 12 years old; is from 0.237 ng/ml to 6.385 ng/ml for a healthy subject from about 12 years old to about 18 years old; is from 0.272 ng/ml to 7.111 ng/ml for a healthy subject from about 18 years old to about 40 years old; is from 0.319 ng/ml to 6.392 ng/ml for a healthy subject from about 40 years old to about 60 years old; is from 0.317 ng/ml to 4.928 ng/ml for a healthy subject over 60 years old.

Additionally or alternatively, the computer readable medium may contain the information that the reference Lyso-Gb3 range for a healthy subject up to about 4 years old is from 0.0441 ng/ml to 1.952 ng/ml; is from 0.223 ng/ml to 2.326 ng/ml for a healthy subject from about 4 years old to about 12 years old; is from 0.138 ng/ml to 2.014 ng/ml for a healthy subject from about 12 years old to about 18 years old; is from 0.145 ng/ml to 1.971 ng/ml for a healthy subject from about 18 years old to about 40 years old; is from 0.085 ng/ml to 2.014 ng/ml for a healthy subject from about 40 years old to about 60 years old; is from 0.118 ng/ml to 1.774 ng/ml for a healthy subject over 60 years old. The computer readable medium may further contain information on gender-dependent reference values. For example, the computer readable medium may contain the information that a suitable reference Lyso-Gb3 range for a healthy female subject up to about 4 years old is from 0.0441 ng/ml to 1.518 ng/ml and/or for a healthy male subject up to about 4 years old is from 0.057 ng/ml to 1.952 ng/ml. The computer readable medium may contain the information that a suitable reference Lyso-Gb3 range for a healthy female subject from about 4 years old to about 12 years old is from 0.262 ng/ml to 2.217 ng/ml and/or for a healthy male subject from about 4 years old to about 12 years old is from 0.223 ng/ml to 2.326 ng/ml.

Upon determination of the level of a plurality of biomarkers (i.e. multiple biomarkers) in the dried blood spot sample or portion thereof, skilled practitioners (e.g., physicians, genetic counsellors, researcher) or the subject may be informed of the result and may or may not be informed of the relevant age dependent reference range for one or more biomarkers which have been determined. Optionally, the result can be cast in a transmittable form that can be communicated or transmitted to other researchers or physicians or genetic counsellors or patients. Such a form can vary and can be tangible (e.g., papers, computer readable media such as floppy disks, compact disks) or intangible (e.g., by means of email, website, or intranet). The result with regard to the presence or absence of a biomarker, and optionally the quantitative amount thereof in the individual tested can be communicated for example by descriptive statements, diagrams, photographs, charts, images or any other visual forms.

A further aspect of the invention relates to a kit of parts for detecting multiple biomarkers indicative for lysosomal storage diseases from a dried blood spot sample, wherein the kit comprises:
an extraction solution comprising 35% to 65% DMSO and 35% to 65% methanol;
an internal standard comprising at least two stable isotope labeled reference biomarkers each indicative for a lysosomal storage disease; and
a set of instructions for performing the method subject of the invention, and/or a set of reference ranges for one or more biomarkers.

The terms “kit of parts” and “kit” as used herein refer to a product containing components necessary for carrying out the methods (e.g., the method for detecting a multiple biomarkers each indicative for a lysosomal storage disease), packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports.

The components comprised in a kit may be sufficient or may not be sufficient for carrying out the specified methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the recited set of components as taught herein (i.e., at least DMSO and methanol), the present kits may also include some or all of solvents, buffers. Examples of solvents of buffers include without limitation histidine-buffers, citrate-buffers, succinate-buffers, acetate-buffers, phosphate-buffers, formate buffers, benzoate buffers, TRIS (Tris(hydroxymethyl)-aminomethan) buffers or maleate buffers, or mixtures thereof. Additionally or alternatively, the kit of parts may include enzymes, detectable labels, detection reagents, and control formulations (positive and/or negative), useful in the method subject of the invention. The terms may be used interchangeably with the term “article of manufacture”, which broadly encompasses any man-made tangible structural product, when used in the present context. Typically, the kits may also include instructions for use thereof, such as on a printed insert or on a computer readable medium. The kit may further comprise documents regarding safety, documents concerning quality assurance and any other information that is commonly provided in kit of parts.

The kit may comprise a set of reference ranges for the plurality of biomarkers that can be detected by the kit. For example, the kit can comprise reference ranges for Lyso-Gb1 and/or Lyso-Gb1 expected for healthy subjects. The presentation particulars of said reference ranges are not limiting in the context of the invention and may therefore be part of the kit in the form of a physical leaflet, a digital storage device, or both.

The kit of parts may comprise DMSO and methanol, which are essential for the extraction solution, in a single container or separate containers that are to be mixed by the user prior to performing the method described herein. The kit or parts may comprise DMSO and methanol to conduct the method described herein a multiple amount of times. The kit of parts may comprise an unused (i.e., fresh, “new”, blank), filter paper or equivalent suitable substrate that is to be used to spot a blood sample on to arrive at a dried blood spot sample. The kit of parts may comprise desiccant to aid in storage of one or more components of the kit, such as the blood spot sample substrate e.g., the filter paper. The kit of parts may comprise a container for temporary storage of one or more dried blood spot samples. The kit of parts may comprise a disposable punching tool to excise and/or isolate the dried blood spot sample or portion thereof from a larger substrate.

As indicated above, the kit of parts may comprise one or more suitable control samples. Without limitation, the kit may therefore comprise as negative control a sample ready or substantially ready for the detection step of the method wherein said sample does not comprise any of the biomarkers indicative for lysosomal storage diseases that are being tested. Without limitation, the kit may therefore comprise as positive control a sample ready or substantially ready for the detection step of the method wherein said sample does comprises one or more of the biomarkers indicative for a lysosomal storage disease in an amount that corresponds to an amount sufficient for the method to indicate that a subject has a lysosomal storage disease. The kit of parts may comprise a single positive control that contains each of the plurality of biomarkers that is to be tested, or may comprise multiple positive controls that each contain a biomarker or a subset of the group of biomarkers that is to be tested by the method described herein.

In certain embodiments, the kit of parts comprises one or more reagents for performing one or more mass spectrometry assay. In certain embodiments, the kit of parts comprises information about one or more transitions that may be detected by the method described herein and guidance on their interpretation. In certain embodiments, the kit of parts comprises one or more reagents for performing an enzymatic activity assay. In certain embodiments, the kit of parts comprises one or more reagents for performing a genetic analysis assay. In such embodiments, the kit of parts may comprise means to extract and/or isolate DNA and/or RNA. Optionally, the kit of parts comprises at least one primer pair for performing a polymerase chain reaction. Optionally, the kit of parts comprises at least a polymerase, preferably a DNA polymerase in an amount sufficient for conducting a polymerase chain reaction.

The term “primer pair” or “amplification primer pair” refers to a combination of two primers which are suited for amplification of a target nucleic acid region (amplicon) from within a nucleic acid of interest by a polymerase-based amplification process. The ability to amplify an amplicon from within the nucleic acid of interest using a primer pair designed to specifically hybridise within the nucleic acid indicates the presence (and optionally quantity) of the nucleic acid in the polymerase-based amplification reaction.

Preferably, the kit of parts comprises at least means to detect the presence of Lyso-Gb1 and Lyso-Gb3 in a dried blood spot sample. More preferably, the kit of parts comprises means to detect the presence of Lyso-Gb1 and Lyso-Gb3 at least 2 times, preferably at least 3 times, preferably at least 4 times, preferably at least 5 times, preferably at least 10 times, preferably at least 15 times, preferably at least 20 times, preferably at least 25 times.

The kit of parts comprises an internal standard. Said internal standard comprises a reference biomarker entity that corresponds to at least one of the biomarkers that are to be detected by the method described herein. Preferably, the internal standard comprises a stable isotope labeled reference biomarker. In particular embodiments, the internal standard comprises a stable isotope labeled Lyso-Gb1 molecule. In particular embodiments, the internal standard comprises a stable isotope labeled Lyso-Gb3 molecule. In preferred embodiments, the kit of parts comprises as a first reference biomarker stable isotope labeled Lyso-Gb1 and as second reference biomarker stable isotope labeled Lyso-Gb3. In further preferred embodiments, the kit of parts comprises as a first reference biomarker stable isotope labeled Lyso-Gb1, as a second reference biomarker isotope labeled Lyso-Gb3, and as a third reference biomarker a stable lysosphingomyelin (Lyso-SPM). In yet further embodiments, the kit of parts comprises as a first reference biomarker stable isotope labeled glucosylsphingosine (Lyso-Gb1), as second reference biomarker stable isotope labeled globotriaosylsphingosine (Lyso-Gb3) biomarker, and as third reference biomarker stable isotope labeled lysosphingomyelin (Lyso-SPM and lyso-galactosylceramide In yet further embodiments, the kit of parts consists essentially of, or consists of a first reference biomarker stable isotope labeled glucosylsphingosine (Lyso-Gb1), a second reference biomarker stable isotope labeled globotriaosylsphingosine (Lyso-Gb3) biomarker, and optionally as third reference biomarker stable isotope labeled lysosphingomyelin (Lyso-SPM).

The physical form or physical representation of the internal standard in the kit of parts is not particularly limiting for the invention. Therefore, the internal standard may be comprised in the kit of parts as liquid, powder, or a combination of a liquid and a powder. Optionally, the internal standard may be lyophilized. In such embodiments, the kit of parts may comprise a suitable liquid or solution that allows reconstitution prior to usage of the internal standard.

The term “lyophilized” as used herein refers to a condition and/or state of a sample, formulation, or product. Lyophilization, also known as freeze-drying or cryodesiccation, is a dehydration process which involves freezing the product without destroying the physical structure of the matter. Lyophilization comprises at least a freezing step and a sublimation step. The sublimation step may comprise two stages of drying: a primary drying step and a secondary drying step. Lyophilization may be used in the manufacturing of pharmaceutical products and intermediates thereof. During freezing, the material is cooled to a temperature wherein the solid, liquid, and gas phases of the material may exist. Active pharmaceutical product ingredients (APIs) may be lyophilized to achieve chemical stability allowing room temperature storage. Advantages of lyophilisation may be but are not limited to improved aseptic handling, enhanced stability of a dry powder, the removal of water without excessive heating of the product, and enhanced product stability in a dry state. Lyophilisation methods and advantages of lyophilisation have been described in the art and are therefore known to a skilled person (e.g., in Adams, Methods Mol Biol, 2007). As used herein, “reconstitution” refers to the process of restoring a dried, lyophilized, dehydrated, or concentrated matter to its original or liquid state by adding a solvent to the lyophilized matter, preferably followed by agitating the solvent-lyophilized matter mixture.

In certain embodiments of the kits described above, the kit provides means for generating an outcome value which is submitted in an online tool such as, but not limited to, a website or a mobile application.

As is evident to a skilled person, the kit of parts may be adapted to correspond to any composition, plurality of biomarkers and lysosomal storage diseases that are envisaged by the distinct embodiments of the method described throughout the present disclosure. The skilled person further appreciates that the different embodiments described herein for the method of detecting multiple biomarkers indicative for lysosomal storage diseases readily apply, where applicable, to the kit of parts described herein, and vice versa.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples.

### EXAMPLES

### Example 1. Combined Analysis of Lyso-Gb-1 and Lyso-Gb3 in Dried Blood Sports by LC Tandem Mass Spectrometry

### Materials and Methods

### Chemicals and Reagents

Globotriaosylsphingosine (Lyso-Gb3) (Purity ≥98%, molecular weight 786 g/mol) and Lyso-glucosylsphingosine (Lyso-Gb1) (Purity ≥98%, molecular weight 462 g/mol) were purchased from Matreya LLC, State College, PA, USA and dissolved in a mixture of Chloroform:Methanol (2:1) to make a 1 mg/mL stock solution. 13C₆-Lyso-Gb3 (Purity ≥98%, molecular weight 791.87 g/mol) was used as internal standard and purchased from Gelb Chem, LLC, Seattle WA.

Analytical chemicals and solvents include Formic acid (Purity 99-100%, density 1.22 kg/L), purchased from VWR Chemicals in France. Acetonitrile UPLC, Water ULC/MS (Purity ≥99%), Methanol (MeOH, Purity 99.98%) ULC/MS-CC/SFC and Isopropanol UPLC (Purity ≥99%). All were purchased from Biosolve chimie SARL in France. Dimethylsulfoxide (DMSO, Purity %, Molar mass 78, 13 g/mol) and Chloroform (Purity 99-99.4%) were purchased from Merck, Sigma Aldrich, Germany. Physiological water (Purity ≥99%), was purchased from Baxter, Switzerland.

### Standards, Internal Standards and Quality Controls

The validation protocol defined by CLSI EP05 and C62 was used. Standard and Quality Control (QC) values were determined based on a literature search of the last 5 years in Pubmed (see discussion). Standards were made by serially dilution of the stock solution with DMSO:MeOH (1:1) at concentrations of 100, 500, 1000, 2000, 10000, 50000 and 100000 ng/ml for Lyso-Gb1 and 20, 50, 200, 800, 8000, 16000 and 40000 ng/ml for Lyso-Gb3. Ten μL of the above dilutions for both Lyso-Gb1 and Lyso-Gb3 were combined and diluted 100 times in a 1:1 ratio washed red blood cells (RBC) and DMSO:Physiological water (52%: 48%). After this dilution, new concentrations were obtained of 1, 5, 10, 20, 100, 500 and 1000 ng/ml for Lyso-Gb1 and 0.2, 0.5, 2, 8, 80, 160 and 400 ng/ml for Lyso-Gb3, used as calibration standards (Table 1). Quality controls (QC) were analogously prepared from the stock solutions (1 mg/mL) at concentrations of 400, 1000, 4000, 20000 and 80000 for Lyso-Gb1 and 120, 300, 1000, 5000 and 20000 ng/ml for lyso-Gb3, followed by dilution with washed RBC, DMSO and physiological water. The obtained quality control concentrations were 4, 10, 40, 200 and 800 ng/ml for Lyso-Gb1 and 1.2, 3, 10, 50, 200 ng/ml for Lyso-Gb3, representing values expected to be close to LOQ, medium and high levels (Table 2). The washed red blood cells were obtained from one healthy person. Whole blood was collected with K3-EDTA as anticoagulant, was washed 3 times with physiological water by centrifugation (Beckan Coulter, Allegra x-15R) at 4000 rpm for 5 minutes. Plasma and white blood cells (WBC) were removed after the first time of centrifugation and physiological water was added to the remaining RBCs (1:1 ratio, Hematocrit of 50%). Afterwards the remaining upper layer was removed, leaving the washed red blood cells for preparation of the Lyso-Gb1 and Lyso-Gb3 mixtures as stated above. 70 μL of each standard or QC level was spotted onto filter paper (PerkinElmer, Turku, Finland) and air-dried for at least 12 hours. All standards and QC levels were stored in zip lock plastic bags at −20° C. with desiccant until further analysis.

TABLE 1
Overview of the Standard concentrations.

| Standard (STD) | STD1 | STD2 | STD3 | STD4 | STD5 | STD6 | STD7 |
|---|---|---|---|---|---|---|---|
| Lyso-Gb1 (ng/ml) | 1 | 5 | 10 | 20 | 100 | 500 | 1000 |
| Lyso-Gb3 (ng/ml) | 0.2 | 0.5 | 2 | 8 | 80 | 160 | 400 |

TABLE 2
Overview of the Quality Control concentrations.

| Quality Concentration | QC1 | QC2 | QC3 | QC4 | QC5 |
|---|---|---|---|---|---|
| Lyso-Gb1 (ng/ml) | 4 | 10 | 40 | 200 | 800 |
| Lyso-Gb3 (ng/ml) | 1.2 | 3 | 10 | 50 | 200 |

### Sample Preparation

Three punches (⅛ inches, punch tool Kangaro, India) of 3.2 mm DBS equal a volume of 9.3 μL. Standards, QCs and blank filter paper were punched into a 96-well microplate (Waters, US-made in Mexico) and 150 μL of extraction solution (DMSO:MeOH: 1:1) with the internal standard 13C6-Lyso-Gb3 was added (20 μl on 10 ml extraction solution. Extraction was obtained by incubation for 20 min at 37° C. on an orbital shaker at 300 rpm (PerkinElmer, DELFIA PlateShake). Afterwards the samples were centrifuged (Beckman Coulter, Allegra x-15R) for 10 min at 4750 rpm and 20° C. to obtain a clear supernatant. 100 μL of the supernatant layer, was transferred to a 96-well microplate (Waters, US-made in Mexico) removing the left over punch and centrifuged for 10 min for additional yet optional purification of the sample before analysis. Two μL of the extracted mixture was injected into the liquid chromatography tandem mass spectrometry (LC-MS/MS) system.

Liquid Chromatography with Tandem Mass Spectrometry

The analysis was performed on a QTRAP5500 (AB Sciex, United States) detector with Nexera X2 LC-30AD ultra-high performance liquid chromatography pumps (Shimadzu Scientific Instruments, Columbia, Maryland). Specific settings for the QTRAP5500 system are provided in Table 3 Electrospray ionization (ESI) in positive mode was used for peak detection. The settings for the Multiple Reaction Monitoring (MRM) transition of 13C6-Lyso-Gb3 were 792.392>282.3 m/z, of Lyso-Gb1 were 462.294>282.3 m/z and for Lyso-Gb3 786.392>282.3 m/z. Separation of the prepared samples was achieved on a C18 column (Acquity UPLC CSH C18 1.7 μm from Waters, USA); 2.1 mm×50 mm) with a column temperature of 40° C. To protect the column from contamination a precolumn (Waters, USA) was used.

TABLE 3
Specific settings used on the QTRAP5500 and LC system.
Compound parameters

| MRM | test | Dwell time (ms) | DP (Volts) | CE (Volts) | CXP (Volts) |
|---|---|---|---|---|---|
| 1 | Lyso-Gb3-IS | 53.0 | 171.0 | 45.0 | 20.0 |
| 2 | Lyso-Gb1 | 53.0 | 171.0 | 45.0 | 20.0 |
| 3 | Lyso-Gb3 | 53.0 | 176.0 | 31.0 | 20.0 |

LC-MS/MS source settings

| Total | Pressure | Needle | Sampling | Cooler | Oven | ESI | Rinsing |
|---|---|---|---|---|---|---|---|
| Flow | Limits | Stroke | speed | Temp | Temp | needle | Volume |
| 0.50 ml/min | 14000 psi | 50 mm | 2.0 μl/sec | 15° C. | 40° C. | 50 mm | 500 μl |

| EP | CUR | CAD | IS | TEM | GS1 | GS2 |
|---|---|---|---|---|---|---|
| 10 V | 35 psi | medium | 5500 V | 600° C. | 60 psi | 50 psi |

MRM: Multiple Reaction Monitoring; ms: milliseconds; DP: declustering potential; CE: capillary electrophoresis; CXP: Collision Cell Exit Potential; psi: Pound-force per square inch; IS: internal standard; LC-MS/MS: Liquid Chromatography with tandem mass spectrometry: EP: Enterance potential; CUR: Curtain gas; IS: Ionspray voltage; TEM: temperature; GS1: ion source gas 1; GS2: ion source gas 2; V: volts.

A gradient elution utilizing 0.1% formic acid in water as solvent A and 0.1% formic acid in 80% acetonitrile and 20% MeOH as solvent B was performed, the flow rate was 0.50 ml/min. The gradient was set at 75% A-25% B), changed gradually to 0% A-100% B at 2.5 min, and returned to initial conditions after 0.1 min. The total run time was equal to 5 min. The washing solution was 30% MeOH: 30% water: 30% Acetonitrile: 10% Isopropranol.

Retention time was set at 1.70-1.72 min for Lyso-Gb3 and 13C6-Lyso-Gb3 and 1.82 min for lyso-Gb1.

### Method Validation

According to the CLSI EP05 and C62 guideline a minimum of 20 individual runs with 3 series of the predetermined samples give rise to 60 analytical results to verify the within-run and between-run precision and accuracy, carry-over and limit of quantification (LOQ). The current validation protocol encompasses In total 33 individual runs resulting in 99 analyses. Statistical analyses were carried out using R statistical soft-ware v2.10.1 (Revolution analytics, Palo Alto, CA, USA).

### Linearity

The analytical linearity of the method was determined by analysis of the 7 calibration standards for Lyso-Gb1 and 3. These standard concentrations were obtained out of the 33 different runs. The linear calibration curve was generated by plotting the ratio of the peak area of the detected biomarker concentration versus the theoretical concentration for each of the increasing standard concentrations. A weighing of 1/x was used. The correlation of the measured results and the target values represents the accuracy of this standard level and needs to be <15% as mentioned in the CLSI EP06 guideline. The method was accepted as being linear within the 95% confidence interval. The slope and intercept did not deviate from 1 and 0, respectively with a certainty of p>0.05. Calculations were made by using the Passing Bablok regression, Spearmans correlation and the Bland-Altman test.

### Precision

Precision, expressed as the percentage coefficients of variance (CV %) was carried out by analyzing the DBS QC levels in triplet for all 33 individual runs replicates for intra-day test and in the total amount of results from the 99 analyses for the inter-day test (100×(the standard deviation/calculated mean)). The precision was determined by comparing the measured and spiked concentrations. CV % requires to be < or equal to 15% as defined in the CLSI EP05 and C₆₂ guidelines. To compare the results obtained with the determined cut-off of 15% a Chi-quadrate-test is used. Significant differences were assumed when P<0.05.

### Accuracy

Accuracy (Bias %) was carried out by analyzing the DBS QC levels in 33 replicates for inter-day test and in triplet for inter-day test ((calculated mean-nominal value)/nominal value×100). The accuracy was determined by comparing the measured and spiked concentrations. It required to be < or equal to 15% of the nominal concentration, following CLSI EP05 and C62 guidelines. To compare the obtained results with the determined cut-off of 15% a Chi-quadrate-test is used. Significance was assumed when P<0.05.

### Carry-Over

A carry-over exclusion analysis was performed by analysis of 6 QC low after QC low levels and comparing them with 5 QC low analysed after QC high levels. The difference between low after high and low after low was calculated. When the value for carry-over is lower than 3 times the standard deviation (SD) of the lowest QC value, defined in the CLSI C62 and EP10 guidelines, then the methodology is free from carry-over.

### Lower Limit of Quantification

The LOQ was assessed by using the signal-to-noise method. The LOQ can be calculated by the auto-integrator of the instrument or manually on a chromatogram printout. The ratio between the peak signal over the noise signal, the signal-to-noise ratio (S/N), should be >10 according to the CLSI E17 and C50 guidelines. To account for any variation between runs, the LOQ of 10 different runs was calculated for each biomarker, the mean is used as the finale and representing LOQ. The used area to calculate the LOQ is defined as the full peak width from starting point until the ending point at baseline.

### Interlaboratory Case Finding

Interlaboratory case finding was performed using 3 blinded DBS samples. The samples contained 1 positive for Lyso-Gb3 (FD), one positive for Lyso-Gb1 (GD) and 1 without elevated biomarkers. Comparison was made using the Z-score, defining the deviation from the expected value, which should lay between 1.96 and −1.96 if there is no clinical significant deviation. To calculate the significant correlation between the results, a Spearman correlation was used.

### Results

### Optimization of the LC-MS MS Method

Preliminary experiments were performed to optimize the mass spectrometric parameters, mobile phases, columns and the extraction solution. The optimized ion source parameters were determined via direct infusion of Lyso-Gb1, Lyso-Gb3 and 13C6-Lyso-Gb3 10 ppm solutions in positive ion mode.

### Analytical Validation

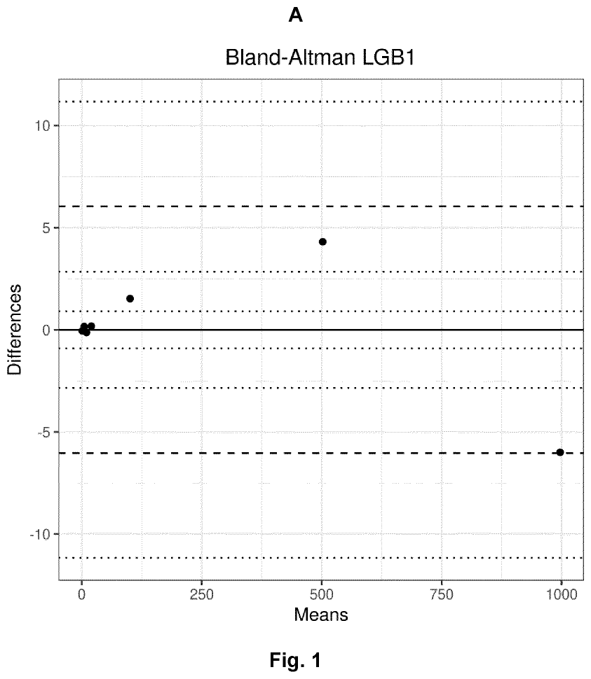

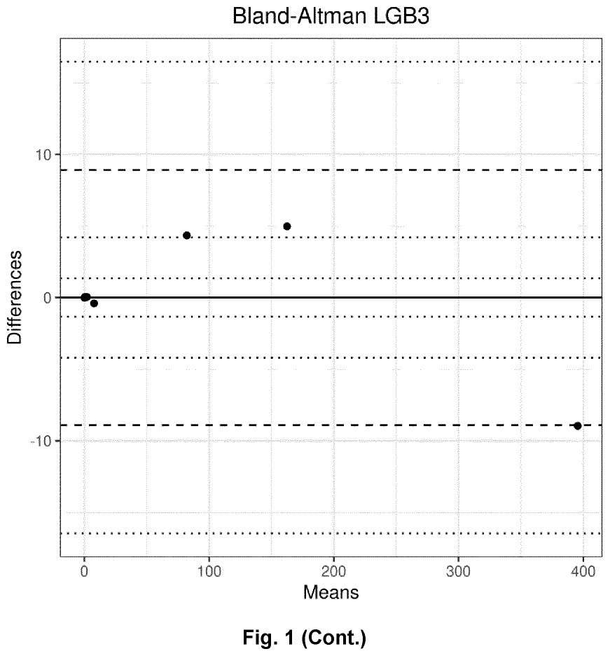

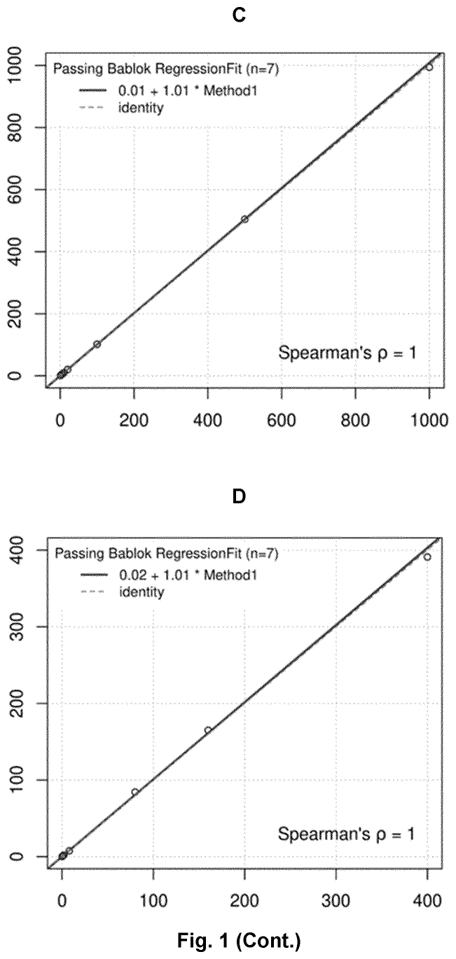

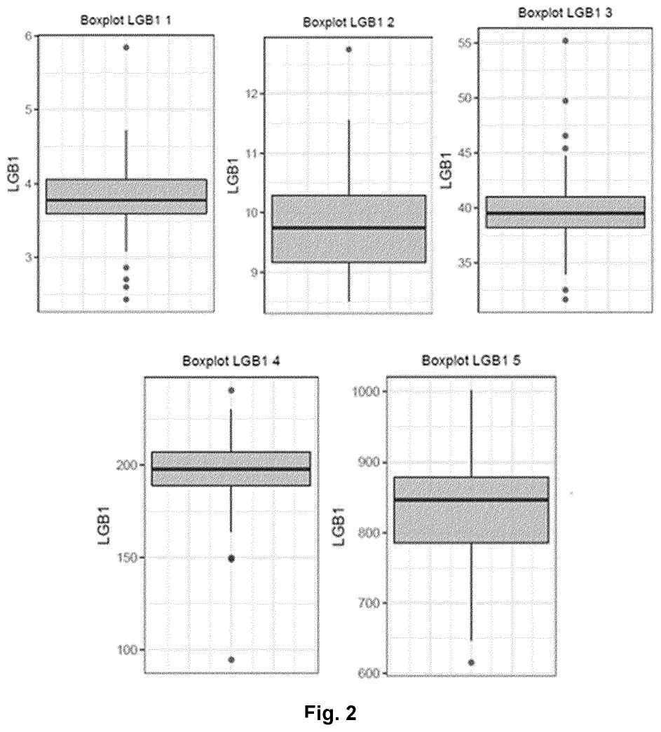

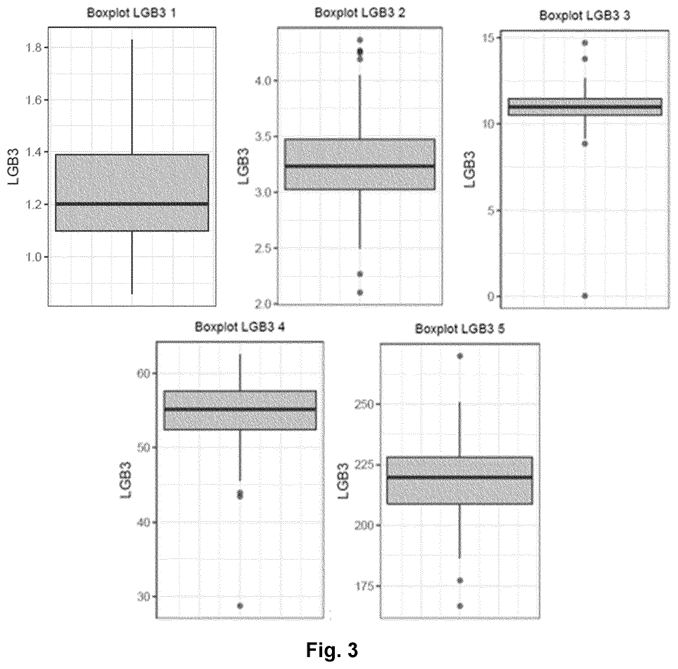

The calibration curve for Lyso-Gb1 and Lyso-Gb3, based on 7 different standard concentration levels, respectively ranged from 1 to 1000 ng/ml and 0.2 to 400 ng/ml (FIG. 1). The calibration curve was analysed in every analytical run (n=33) and was linear with a supporting Passing BaBlok regression result, R²=1 and Spearman's p=1, significant with <0.001), The obtained regression equation for Lyso-Gb1 was defined as y=1.01x+0.01 and for Lyso-Gb3 y=1.01x+0.02.

Data for intra-assay and inter-assay accuracy and precision of Lyso-Gb1 and Lyso-Gb3 were determined based on the QC-values and reported in Table 4 and 5. For all QC values the accuracy and precision were <15%

TABLE 4
Results for the precision, expressed as
the percent of coefficient of variance.

| | | Precision, CV % | Precision, CV % |
|---|---|---|---|
| | QC level | Intra-assay | Inter-assay |
| Lyso- | QC 1 | 10.3 | 6.1 |
| Gb1 | QC 2 | 6.4 | 4.8 |
| | QC 3 | 6.3 | 5.4 |
| | QC 4 | 7 | 6 |
| | QC 5 | 7 | 4.6 |
| Lyso- | QC 1 | 14.5 | 6.5 |
| Gb3 | QC 2 | 12.5 | 4.3 |
| | QC 3 | 11.4 | 6.3 |
| | QC 4 | 6.3 | 5.2 |
| | QC 5 | 6.3 | 3.8 |

QC: Quality control; CV %: percent of coefficient of variance.

TABLE 5
Results for the accuracy, expressed as
the percent of coefficient of variance.

| | | Accuracy, RE % |
|---|---|---|
| | QC level | Inter-assay |
| Lyso- | QC 1 | −5 |
| Gb1 | QC 2 | −1.8 |
| | QC 3 | −0.9 |
| | QC 4 | −1.9 |
| | QC 5 | 4.5 |
| Lyso- | QC 1 | 3.3 |
| Gb3 | QC 2 | 8.7 |
| | QC 3 | 8.8 |
| | QC 4 | 9.1 |
| | QC 5 | 9.3 |

QC: Quality control; CV %: percent of coefficient of variance.

Carry over was determined by the analysis of the difference in signal of low level standard analyzed after a high level of standard (carry-over) and was in absolute numbers 0.65 ng/ml for Lyso-Gb1 and 0.53 ng/ml Lyso-Gb3. The cut-off, defined by 3 times the SD of the lowest QC is respectively 1.37 ng/ml and 1.03 ng/ml (Table 6). Both results are significantly lower than their defined cut-off values. Concluding that the method is not exposed to unwanted carry-over between samples or resulting from device carry-over.

TABLE 6
Results of the carry-over analysis.

| | | Low_low | High_low | Carry-over | |
|---|---|---|---|---|---|
| | Cut-off | mean | mean | Absolute | Conclusion |
| Lyso- | 1.37 | 3.81 | 4.46 | 0.65 | Below cut-off |
| Gb1 | | | | | |
| Lyso- | 1.033 | 1.40 | 1.92 | 0.53 | Below cut-off |
| Gb3 | | | | | |

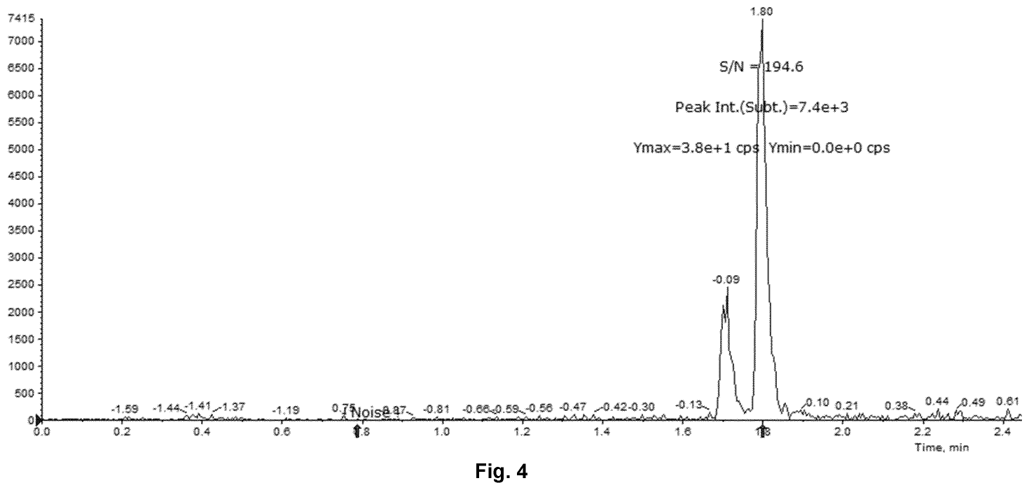

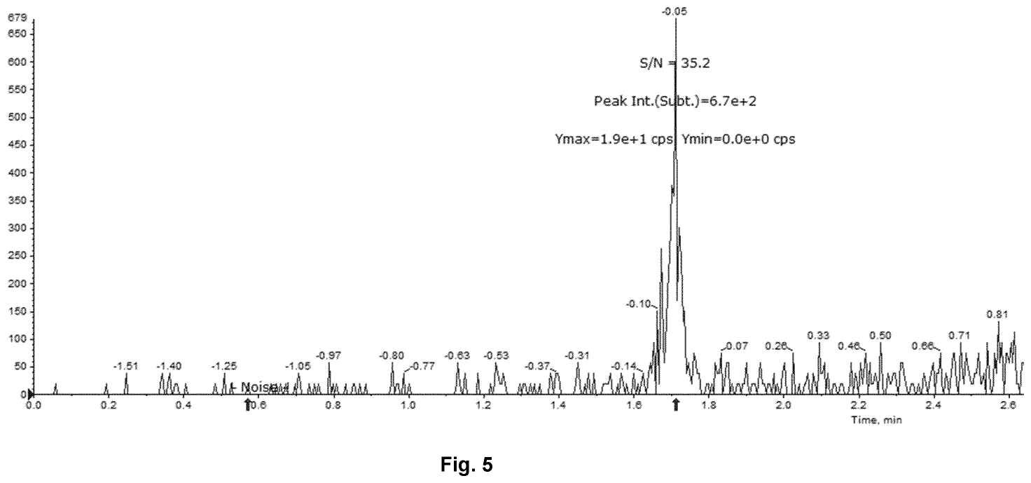

The signal-to-noise ratio was found to be 151.23 at the concentration level of 1 ng/ml and 22.08 at concentration level 0.2 ng/ml respectively for Lyso-Gb1 and Lyso-Gb3. Both above 10 and thus defining the significant increase of the measured value versus the detectable noise in the sample (FIGS. 4 and 5).

The comparison of the Interlaboratory case finding was made using the Z values, defining the variation between the samples were within the defined range, results were represented in Table 7. Most results were inside this defined range. Two samples (Lyso-Gb1 sample 2 and 3) had a variation outside the specified range which is expected due to the clinical significant variation between the labs. Namely each lab was using their own detection method, differences in the methods cause variation in the results. But wat is the most relevant looking at the results of both samples, is the clinical conclusions comparing with the defined cut-off values used by the external lab (6.8 ng/ml for Lyso-Gb1 and 1.8 ng/ml for Lyso-Gb3) were identical (4,56). For the value of inter-run precision, the calculated CV % from QC2, which needs to be <15% as described before, were used, namely 7.9 and 13.2 respectively. Results of both labs were compared and had a significant correlation that was suggestive to be linear (Spearman 1, r2 1, P<0.001). Results are represented in Table 5.

TABLE 7
Data of comparison with external samples.

| | Lyso-Gb1 | Lyso-Gb1 | | Lyso-Gb3 | Lyso-Gb3 | |
|---|---|---|---|---|---|---|
| | Intern | Extern | | Intern | Extern | |
| Sample | (ng/ml) | (ng/ml) | Z-value | (ng/ml) | (ng/ml) | Z-Value |
| 1 | 5.7 | 6.2 | 0.788 | 1.63 | 1.4 | 0.755 |
| 2 | 22.5 | 37.3 | 5.89 | 0.86 | 0.8 | 0.375 |
| 3 | 0 | 1.2 | 1884.359 | 35.3 | 33.3 | 0.305 |

| | | | | | Significance | |
|---|---|---|---|---|---|---|
| | Intercept | Slope | Spearman | R2 | correlation | Kappa |
| Lyso-Gb1 | −0.75 | 0.62 | 1 | 1 | <0.001 | 0.083 |
| Lyso-Gb3 | 0.01 | 1.06 | 1 | 1 | <0.001 | 0.083 |

### Example 2. Comparison of the Extraction Solution of the Present Invention with an Extraction Solution Reported in the Art

Malvagia and colleagues (Clin Chem Lab Med, 2021) reported a method to conduct Lyso-Gb3 measurements starting from a dried blood spot sample. The method relies on the use of an extraction solution of 100% methanol. To provide an unambiguous and direct comparison of the performance of both extraction solutions for detection of both Lyso-Gb1 and Lyso-Gb3 in a single detection run, the inventors have performed the complete method with 100% methanol (Tables 8 and 9) as extraction solution and the preferred extraction solution of 50% methanol+50% DMSO (Tables 10 and 11).

Except for the extraction solution, all other parameters between the experiments were identical. 3 spots of dried blood spot samples were incubated for each condition in one of the extraction solutions and incubated for 20 minutes at 37° C. prior to centrifugation at 4750 rpm for 10 minutes in an Allegra x-15R device. Solvent A used contained 0.1% formic acid in water and solvent B 0.1% formic acid in 20% methanol and 80% acetonitrile. A stable flow rate of about 0.50 ml/min was maintained. The gradient was set at 75% A-25% B at the start of the chromatography process which was gradually changed to 0% A-100% B by 2.5 min after starting the process. For Lyso-Gb3 the 786.3 m/z parent ion and 282.3 m/z was measured. For Lyso-Gb1 the 462.294 m/z parent ion and 282.3 m/z daughter ion was measured. An injection volume of 2 μl was used for the QTRAP5500 mass spectrometer.

The coefficients of variation for both experiments are shown in Tables 12 and 13.

TABLE 8
Lyso-Gb1 measurements using 50% methanol + 50% DMSO as extraction solution.

| Index | Sample [symbol] | Sample [symbol] | Sample [symbol] | IS | Compon [symbol] | IS Name | Compon [symbol] | Outlier R [symbol] |
|---|---|---|---|---|---|---|---|---|
| 3 | solvent | | Solvent | False | Igb1_282 | Igb3-is | | |
| 8 | bl | | Blank | False | Igb1_282 | Igb3-is | | |
| 13 | strd1 | | Standard | False | Igb1_282 | Igb3-is | | Accuracy |
| 18 | strd2 | | Standard | False | Igb1_282 | Igb3-is | | |
| 23 | strd3 | | Standard | False | Igb1_282 | Igb3-is | | |
| 28 | strd4 | | Standard | False | Igb1_282 | Igb3-is | | |
| 33 | strd5 | | Standard | False | Igb1_282 | Igb3-is | | |
| 38 | strd6 | | Standard | False | Igb1_282 | Igb3-is | | |
| 43 | strd7 | | Standard | False | Igb1_282 | Igb3-is | | |
| 48 | solvent | | Solvent | False | Igb1_282 | Igb3-is | | |
| 53 | QC1 | | Quality [symbol] | False | Igb1_282 | Igb3-is | | Accuracy |
| 58 | QC2 | | Quality [symbol] | False | Igb1_282 | Igb3-is | | |
| 63 | QC3 | | Quality [symbol] | False | Igb1_282 | Igb3-is | | |
| 68 | QC4 | | Quality [symbol] | False | Igb1_282 | Igb3-is | | |
| 73 | QC5 | | Quality [symbol] | False | Igb1_282 | Igb3-is | | |
| 78 | solvent | | Solvent | False | Igb1_282 | Igb3-is | | |
| 83 | QC1 | | Quality [symbol] | False | Igb1_282 | Igb3-is | | |
| 88 | QC2 | | Quality [symbol] | False | Igb1_282 | Igb3-is | | |
| 93 | QC3 | | Quality [symbol] | False | Igb1_282 | Igb3-is | | |
| 98 | QC4 | | Quality [symbol] | False | Igb1_282 | Igb3-is | | |
| 103 | QC5 | | Quality [symbol] | False | Igb1_282 | Igb3-is | | |
| 108 | Solvent | | Solvent | False | Igb1_282 | Igb3-is | | |
| 113 | QC1 | | Quality [symbol] | False | Igb1_282 | Igb3-is | | |
| 118 | QC2 | | Quality [symbol] | False | Igb1_282 | Igb3-is | | |
| 123 | QC3 | | Quality [symbol] | False | Igb1_282 | Igb3-is | | |
| 128 | QC4 | | Quality [symbol] | False | Igb1_282 | Igb3-is | | |
| 133 | QC5 | | Quality [symbol] | False | Igb1_282 | Igb3-is | | |
| 138 | Solvent | | Solvent | False | Igb1_282 | Igb3-is | | |

| Index | Actual C [symbol] | Area | Height | Retentio [symbol] | Width at [symbol] | Used | Calculat [symbol] | Accuracy |
|---|---|---|---|---|---|---|---|---|
| 3 | N/A | 14515.9 | 3995.7 | 1.9395 | 0.05044 | True | N/A | N/A |
| 8 | N/A | 1038.98 | 360.042 | 2.00082 | 0.04519 | True | <0 | N/A |
| 13 | 1 | 6115.99 | 2172.06 | 2.00643 | 0.04375 | True | 0.77671 | 77.671 |
| 18 | 5 | 10674.6 | 3819.73 | 2.00524 | 0.04249 | True | 5.57682 | 111.536 |
| 23 | 10 | 18572.5 | 7113.47 | 2.00244 | 0.04028 | True | 9.39077 | 93.9077 |
| 28 | 20 | 5315.27 | 1903.64 | 2.00042 | 0.04326 | True | 21.0102 | 105.051 |
| 33 | 100 | 12626.9 | 4683.59 | 2.00426 | 0.04083 | True | 114.737 | 114.737 |
| 38 | 500 | 312718 | 113096 | 2.00231 | 0.04145 | True | 486.459 | 97.2919 |
| 43 | 1000 | 1122300 | 400969 | 2.00522 | 0.04195 | True | 998.049 | 99.8049 |
| 48 | N/A | 2692.48 | 798.699 | 2.00949 | 0.05048 | True | N/A | N/A |
| 53 | 4 | 7975.78 | 2961.24 | 2.00888 | 0.04104 | True | 2.59445 | 64.8612 |
| 58 | 10 | 17799.1 | 6471.37 | 2.00611 | 0.04217 | True | 8.78216 | 87.8216 |
| 63 | 40 | 65634.9 | 23872 | 2.00135 | 0.042 | True | 38.6532 | 96.633 |
| 68 | 200 | 328800 | 119288 | 1.99827 | 0.04158 | True | 181.5 | 90.7502 |
| 73 | 800 | 1380370 | 490237 | 2.00304 | 0.0425 | True | 766.083 | 95.7604 |
| 78 | N/A | 3294.57 | 1098.86 | 1.99682 | 0.04662 | True | N/A | N/A |
| 83 | 4 | 7759.78 | 2775.89 | 1.99986 | 0.04307 | True | 4.09328 | 102.332 |
| 88 | 10 | 20143.8 | 7408.53 | 2.00621 | 0.04156 | True | 10.2932 | 102.932 |
| 93 | 40 | 63816.6 | 23161 | 2.01752 | 0.04172 | True | 37.4582 | 93.6456 |
| 98 | 200 | 292588 | 105518 | 2.00851 | 0.04176 | True | 179.998 | 89.9988 |
| 103 | 800 | 1213537 | 430652 | 2.00869 | 0.04254 | True | 753.49 | 94.1862 |
| 108 | N/A | 4273.93 | 1072.09 | 2.00616 | 0.05182 | True | N/A | N/A |
| 113 | 4 | 7726.03 | 2891.47 | 2.00844 | 0.04174 | True | 3.47614 | 86.9034 |
| 118 | 10 | 13875.8 | 5142.14 | 2.00302 | 0.0421 | True | 9.02259 | 90.2259 |
| 123 | 40 | 55912.8 | 20228.3 | 2.00296 | 0.04211 | True | 36.4869 | 91.2172 |
| 128 | 200 | 310164 | 108809 | 2.00826 | 0.0431 | True | 191.615 | 95.8073 |
| 133 | 800 | 1175204 | 408825 | 2.00897 | 0.04341 | True | 784.818 | 98.1022 |
| 138 | N/A | 4077.42 | 988.577 | 2.00531 | 0.05402 | True | N/A | N/A |

[symbol]

indicates data missing or illegible when filed

TABLE 9
Lyso-Gb3 measurements using 50% methanol + 50% DMSO as extraction solution.

| Index | Sample [symbol] | Sample [symbol] | Sample [symbol] | IS | Compon [symbol] | IS Name | Compon [symbol] | Outlier R [symbol] |
|---|---|---|---|---|---|---|---|---|
| 1 | solvent | | Solvent | False | Igb3_282 | Igb3-is | | |
| 6 | bl | | Blank | False | Igb3_282 | Igb3-is | | |
| 11 | strd1 | | Standard | False | Igb3_282 | Igb3-is | | Accuracy |
| 16 | strd2 | | Standard | False | Igb3_282 | Igb3-is | | |
| 21 | strd3 | | Standard | False | Igb3_282 | Igb3-is | | |
| 26 | strd4 | | Standard | False | Igb3_282 | Igb3-is | | |
| 31 | strd5 | | Standard | False | Igb3_282 | Igb3-is | | |
| 36 | strd6 | | Standard | False | Igb3_282 | Igb3-is | | |
| 41 | strd7 | | Standard | False | Igb3_282 | Igb3-is | | |
| 46 | solvent | | Solvent | False | Igb3_282 | Igb3-is | | |
| 51 | QC1 | | Quality [symbol] | False | Igb3_282 | Igb3-is | | Accuracy |
| 56 | QC2 | | Quality [symbol] | False | Igb3_282 | Igb3-is | | |
| 61 | QC3 | | Quality [symbol] | False | Igb3_282 | Igb3-is | | |
| 66 | QC4 | | Quality [symbol] | False | Igb3_282 | Igb3-is | | |
| 71 | QC5 | | Quality [symbol] | False | Igb3_282 | Igb3-is | | Accuracy |
| 76 | solvent | | Solvent | False | Igb3_282 | Igb3-is | | |
| 81 | QC1 | | Quality [symbol] | False | Igb3_282 | Igb3-is | | |
| 86 | QC2 | | Quality [symbol] | False | Igb3_282 | Igb3-is | | |
| 91 | QC3 | | Quality [symbol] | False | Igb3_282 | Igb3-is | | |
| 96 | QC4 | | Quality [symbol] | False | Igb3_282 | Igb3-is | | |
| 101 | QC5 | | Quality [symbol] | False | Igb3_282 | Igb3-is | | Accuracy |
| 106 | Solvent | | Solvent | False | Igb3_282 | Igb3-is | | |
| 111 | QC1 | | Quality [symbol] | False | Igb3_282 | Igb3-is | | Accuracy |
| 116 | QC2 | | Quality [symbol] | False | Igb3_282 | Igb3-is | | |
| 121 | QC3 | | Unknowr | False | Igb3_282 | Igb3-is | | |
| 126 | QC4 | | Quality [symbol] | False | Igb3_282 | Igb3-is | | |
| 131 | QC5 | | Quality [symbol] | False | Igb3_282 | Igb3-is | | |
| 136 | Solvent | | Solvent | False | Igb3_282 | Igb3-is | | |

| | | | | | Width | | | |
|---|---|---|---|---|---|---|---|---|
| Index | Actual C [symbol] | Area | Height | Retentio [symbol] | at [symbol] | Used | Calculat [symbol] | Accuracy |
| 1 | N/A | N/A | N/A | N/A | N/A | True | N/A | N/A |
| 6 | N/A | 740.823 | 271.389 | 1.90317 | 0.04258 | True | <0 | N/A |
| 11 | 0.2 | 1168.95 | 399.886 | 1.89676 | 0.03944 | True | 0.15304 | 76.5202 |
| 16 | 0.5 | 1126.76 | 373.125 | 1.89859 | 0.04252 | True | 0.5387 | 107.74 |
| 21 | 2 | 2649.27 | 913.857 | 1.89724 | 0.04321 | True | 2.25041 | 112.521 |
| 26 | 8 | 8470.44 | 2697.17 | 1.90577 | 0.04684 | True | 8.04475 | 100.559 |
| 31 | 80 | 80706.1 | 27465.1 | 1.89908 | 0.04463 | True | 84.4601 | 105.575 |
| 36 | 160 | 124244 | 42027.8 | 1.90595 | 0.04411 | True | 155.391 | 97.1196 |
| 41 | 400 | 325387 | 108694 | 1.8955 | 0.04438 | True | 399.862 | 99.9654 |
| 46 | N/A | 873.805 | 292.811 | 1.89485 | 0.04612 | True | N/A | N/A |
| 51 | 0.9 | 1978.01 | 649.722 | 1.89966 | 0.04357 | True | 1.87442 | 208.269 |
| 56 | 3 | 2950.17 | 977.242 | 1.89588 | 0.04391 | True | 2.78278 | 92.7595 |
| 61 | 10 | 10077.6 | 3316.33 | 1.89769 | 0.0448 | True | 11.0762 | 110.762 |
| 66 | 50 | 49627 | 16563.9 | 1.90412 | 0.04475 | True | 54.32 | 108.64 |
| 71 | 200 | 190721 | 63096.9 | 1.89768 | 0.04512 | True | 234.454 | 117.227 |
| 76 | N/A | N/A | N/A | N/A | N/A | True | N/A | N/A |
| 81 | 0.9 | 1828.3 | 684.27 | 1.90263 | 0.04167 | True | 0.91472 | 101.635 |
| 86 | 3 | 4034.23 | 1342.69 | 1.90394 | 0.04521 | True | 3.14271 | 104.757 |
| 91 | 10 | 10909.4 | 3695.14 | 1.90412 | 0.04481 | True | 10.8447 | 108.447 |
| 96 | 50 | 39295.8 | 13373.3 | 1.90532 | 0.04418 | True | 51.3135 | 102.627 |
| 101 | 200 | 202450 | 66535.6 | 1.90298 | 0.0452 | True | 231.648 | 115.824 |
| 106 | N/A | N/A | N/A | N/A | N/A | True | N/A | N/A |
| 111 | 0.9 | 2263.43 | 660.452 | 1.90458 | 0.04788 | True | 1.4289 | 158.766 |
| 116 | 3 | 3484.99 | 1173.51 | 1.89966 | 0.0426 | True | 3.43613 | 114.538 |
| 121 | N/A | 12176.1 | 4038.3 | 1.90003 | 0.04422 | True | 12.6305 | N/A |
| 126 | 50 | 48884.9 | 16127.8 | 1.90079 | 0.04552 | True | 51.3226 | 102.645 |
| 131 | 200 | 185864 | 62967.1 | 1.90542 | 0.04434 | True | 213.845 | 106.922 |
| 136 | N/A | N/A | N/A | N/A | N/A | True | N/A | N/A |

[symbol]

indicates data missing or illegible when filed

TABLE 10
Lyso-Gb1 measurements using 100% methanol as extraction solution.

| Index | Sample [symbol] | Sample ID | Sample T [symbol] | IS | Compone [symbol] | IS Name | Compone [symbol] | Outlier R [symbol] |
|---|---|---|---|---|---|---|---|---|
| 3 | solvent | | Solvent | False | Igb1_282 | Igb3-is | | |
| 8 | bl | | Blank | False | Igb1_282 | Igb3-is | | |
| 13 | strd1 | | Standard | False | Igb1_282 | Igb3-is | | |
| 18 | strd2 | | Standard | False | Igb1_282 | Igb3-is | | |
| 23 | strd3 | | Standard | False | Igb1_282 | Igb3-is | | |
| 28 | strd4 | | Standard | False | Igb1_282 | Igb3-is | | |
| 33 | strd5 | | Standard | False | Igb1_282 | Igb3-is | | |
| 38 | strd6 | | Standard | False | Igb1_282 | Igb3-is | | Accuracy |
| 43 | strd7 | | Standard | False | Igb1_282 | Igb3-is | | |
| 48 | solvent | | Solvent | False | Igb1_282 | Igb3-is | | |
| 53 | QC1 | | Quality [symbol] | False | Igb1_282 | Igb3-is | | |
| 58 | QC2 | | Quality [symbol] | False | Igb1_282 | Igb3-is | | |
| 63 | QC3 | | Quality [symbol] | False | Igb1_282 | Igb3-is | | |
| 68 | QC4 | | Quality [symbol] | False | Igb1_282 | Igb3-is | | |
| 73 | QC5 | | Quality [symbol] | False | Igb1_282 | Igb3-is | | Accuracy |
| 78 | solvent | | Solvent | False | Igb1_282 | Igb3-is | | |
| 83 | QC1 | | Quality [symbol] | False | Igb1_282 | Igb3-is | | Accuracy |
| 88 | QC2 | | Quality [symbol] | False | Igb1_282 | Igb3-is | | |
| 93 | QC3 | | Quality [symbol] | False | Igb1_282 | Igb3-is | | |
| 98 | QC4 | | Quality [symbol] | False | Igb1_282 | Igb3-is | | |
| 103 | QC5 | | Quality [symbol] | False | Igb1_282 | Igb3-is | | Accuracy |
| 108 | solvent | | Solvent | False | Igb1_282 | Igb3-is | | |
| 113 | QC1 | | Quality [symbol] | False | Igb1_282 | Igb3-is | | Accuracy |
| 118 | QC2 | | Quality [symbol] | False | Igb1_282 | Igb3-is | | |
| 123 | QC3 | | Quality [symbol] | False | Igb1_282 | Igb3-is | | |
| 128 | QC4 | | Quality [symbol] | False | Igb1_282 | Igb3-is | | |
| 133 | QC5 | | Quality [symbol] | False | Igb1_282 | Igb3-is | | Accuracy |
| 138 | Solvent | | Solvent | False | Igb1_282 | Igb3-is | | |

| | | | | | Width | | | |
|---|---|---|---|---|---|---|---|---|
| Index | Actual Co [symbol] | Area | Height | Retention | at [symbol] | Used | Calculate [symbol] | Accuracy |
| 3 | N/A | 269.3456 | 136.9964 | 1.77258 | 0.030654 | True | N/A | N/A |
| 8 | N/A | 4018.35 | 1675.233 | 1.691766 | 0.037809 | True | <0 | N/A |
| 13 | 1 | 4697.478 | 2132.75 | 1.77645 | 0.034354 | True | 0.811112 | 81.11125 |
| 18 | 5 | 11021.39 | 4718.466 | 1.782013 | 0.03635 | True | 5.540601 | 110.812 |
| 23 | 10 | 16201.81 | 6949.738 | 1.77753 | 0.035596 | True | 8.785105 | 87.85105 |
| 28 | 20 | 33943.31 | 14036.67 | 1.781001 | 0.036617 | True | 21.79652 | 108.9826 |
| 33 | 100 | 136130.7 | 55865.84 | 1.784635 | 0.036539 | True | 98.71775 | 98.71775 |
| 38 | 500 | 741730.8 | 306272.5 | 1.777121 | 0.035919 | True | 624.9043 | 124.9809 |
| 43 | 1000 | 1082679 | 439197.3 | 1.775659 | 0.036502 | True | 875.4447 | 87.54447 |
| 48 | N/A | 801.7828 | 261.506 | 1.768705 | 0.047592 | True | N/A | N/A |
| 53 | 4 | 8080.017 | 3674.244 | 1.779237 | 0.033483 | True | 3.688905 | 92.22262 |
| 58 | 10 | 15721.56 | 6700.966 | 1.783051 | 0.036061 | True | 10.6032 | 106.032 |
| 63 | 40 | 54153.84 | 22546.48 | 1.778578 | 0.035865 | True | 44.56489 | 111.4122 |
| 68 | 200 | 258201.9 | 97834.66 | 1.781998 | 0.0399 | True | 198.6211 | 99.31053 |
| 73 | 800 | 1181752 | 484572.1 | 1.784527 | 0.036158 | True | 1095.447 | 136.9309 |
| 78 | N/A | 961.665 | 304.3525 | 1.771664 | 0.049854 | True | N/A | N/A |
| 83 | 4 | 6849.138 | 2960.458 | 1.777259 | 0.035406 | True | 3.285351 | 82.13377 |
| 88 | 10 | 15958.31 | 6803.416 | 1.776523 | 0.036451 | True | 11.15063 | 111.5063 |
| 93 | 40 | 54535.4 | 23169.58 | 1.783353 | 0.035731 | True | 43.81954 | 109.5489 |
| 98 | 200 | 243219.8 | 99781.77 | 1.782105 | 0.036261 | True | 208.1069 | 104.0534 |
| 103 | 800 | 1110150 | 459050.7 | 1.780514 | 0.036013 | True | 994.4166 | 124.3021 |
| 108 | N/A | 1069.727 | 336.6203 | 1.775981 | 0.044353 | True | N/A | N/A |
| 113 | 4 | 6692.986 | 2999.845 | 1.785021 | 0.034372 | True | 2.60886 | 65.22151 |
| 118 | 10 | 15227.51 | 6464.167 | 1.777834 | 0.035969 | True | 10.91508 | 109.1508 |
| 123 | 40 | 47682.58 | 19803.82 | 1.781657 | 0.036507 | True | 42.46611 | 106.1653 |
| 128 | 200 | 236749.3 | 97659.22 | 1.78046 | 0.035843 | True | 216.3389 | 108.1694 |
| 133 | 800 | 1046279 | 422302.6 | 1.778508 | 0.037015 | True | 1085.918 | 135.7397 |
| 138 | N/A | 916.4999 | 349.8778 | 1.777832 | 0.038788 | True | N/A | N/A |

[symbol]

indicates data missing or illegible when filed

TABLE 11
Lyso-Gb3 measurements using 100% methanol as extraction solution.

| Index | Sample N [symbol] | Sample ID | Sample T [symbol] | IS | Compone [symbol] | IS Name | Compone [symbol] | Outlier R [symbol] |
|---|---|---|---|---|---|---|---|---|
| 1 | solvent | | Solvent | False | Igb3_282 | Igb3-is | | |
| 6 | bl | | Blank | False | Igb3_282 | Igb3-is | | |
| 11 | strd1 | | Standard | False | Igb3_282 | Igb3-is | | Accuracy |
| 16 | strd2 | | Standard | False | Igb3_282 | Igb3-is | | Accuracy |
| 21 | strd3 | | Standard | False | Igb3_282 | Igb3-is | | |
| 26 | strd4 | | Standard | False | Igb3_282 | Igb3-is | | |
| 31 | strd5 | | Standard | False | Igb3_282 | Igb3-is | | |
| 36 | strd6 | | Standard | False | Igb3_282 | Igb3-is | | Accuracy |
| 41 | strd7 | | Standard | False | Igb3_282 | Igb3-is | | |
| 46 | solvent | | Solvent | False | Igb3_282 | Igb3-is | | |
| 51 | QC1 | | Quality [symbol] | False | Igb3_282 | Igb3-is | | Accuracy |
| 56 | QC2 | | Quality [symbol] | False | Igb3_282 | Igb3-is | | Accuracy |
| 61 | QC3 | | Quality [symbol] | False | Igb3_282 | Igb3-is | | Accuracy |
| 66 | QC4 | | Quality [symbol] | False | Igb3_282 | Igb3-is | | |
| 71 | QC5 | | Quality [symbol] | False | Igb3_282 | Igb3-is | | Accuracy |
| 76 | solvent | | Solvent | False | Igb3_282 | Igb3-is | | |
| 81 | QC1 | | Quality [symbol] | False | Igb3_282 | Igb3-is | | |
| 86 | QC2 | | Quality [symbol] | False | Igb3_282 | Igb3-is | | Accuracy |
| 91 | QC3 | | Quality [symbol] | False | Igb3_282 | Igb3-is | | |
| 96 | QC4 | | Quality [symbol] | False | Igb3_282 | Igb3-is | | |
| 101 | QC5 | | Quality [symbol] | False | Igb3_282 | Igb3-is | | Accuracy |
| 106 | solvent | | Solvent | False | Igb3_282 | Igb3-is | | |
| 111 | QC1 | | Quality [symbol] | False | Igb3_282 | Igb3-is | | |
| 116 | QC2 | | Quality [symbol] | False | Igb3_282 | Igb3-is | | |
| 121 | QC3 | | Quality [symbol] | False | Igb3_282 | Igb3-is | | |
| 126 | QC4 | | Quality [symbol] | False | Igb3_282 | Igb3-is | | Accuracy |
| 131 | QC5 | | Quality [symbol] | False | Igb3_282 | Igb3-is | | Accuracy |
| 136 | Solvent | | Solvent | False | Igb3_282 | Igb3-is | | |

| Index | Actual Co [symbol] | Area | Height | Retention | Width at [symbol] | Used | Calculate [symbol] | Accuracy |
|---|---|---|---|---|---|---|---|---|
| 1 | N/A | N/A | N/A | N/A | N/A | True | N/A | N/A |
| 6 | N/A | 1101.493 | 419.415 | 1.697433 | 0.038825 | True | <0 | N/A |
| 11 | 0.1 | 1190.983 | 384.4346 | 1.689617 | 0.042923 | True | <0 | N/A |
| 16 | 0.5 | 1982.306 | 584.9653 | 1.688773 | 0.04731 | True | 1.202559 | 240.5119 |
| 21 | 2 | 2738.176 | 891.0926 | 1.690058 | 0.041462 | True | 2.216297 | 110.8149 |
| 26 | 8 | 6724.029 | 2407.243 | 1.693581 | 0.04074 | True | 8.696725 | 108.7091 |
| 31 | 80 | 51439.45 | 20339.58 | 1.695698 | 0.037925 | True | 83.1148 | 103.8935 |
| 36 | 160 | 104766.1 | 41185.07 | 1.690228 | 0.037195 | True | 194.7569 | 121.7231 |
| 41 | 400 | 201401.3 | 80253.52 | 1.687199 | 0.037002 | True | 360.6885 | 90.17213 |
| 46 | N/A | N/A | N/A | N/A | N/A | True | N/A | N/A |
| 51 | 0.9 | 2012.017 | 662.9584 | 1.690659 | 0.042417 | True | 1.478092 | 164.2324 |
| 56 | 3 | 3184.531 | 1270.361 | 1.693491 | 0.035737 | True | 3.906473 | 130.2158 |
| 61 | 10 | 7421.888 | 2921.965 | 1.691245 | 0.036926 | True | 12.27568 | 122.7568 |
| 66 | 50 | 31055.37 | 11585.48 | 1.69425 | 0.040013 | True | 51.65598 | 103.312 |
| 71 | 200 | 133900.6 | 53534.69 | 1.697241 | 0.037015 | True | 274.2063 | 137.1032 |
| 76 | N/A | N/A | N/A | N/A | N/A | True | N/A | N/A |
| 81 | 0.9 | 1529 | 535.0526 | 1.688043 | 0.039259 | True | 0.899689 | 99.96541 |
| 86 | 3 | 3409.424 | 1253.53 | 1.687872 | 0.040846 | True | 4.499228 | 149.9743 |
| 91 | 10 | 7066.118 | 2842.142 | 1.695908 | 0.035582 | True | 11.27212 | 112.7212 |
| 96 | 50 | 30439.8 | 11556.91 | 1.692325 | 0.039225 | True | 56.47573 | 112.9515 |
| 101 | 200 | 130724 | 51246.29 | 1.692283 | 0.038146 | True | 258.6325 | 129.3163 |
| 106 | N/A | N/A | N/A | N/A | N/A | True | N/A | N/A |
| 111 | 0.9 | 1617.972 | 511.6543 | 1.694944 | 0.04571 | True | 0.791537 | 87.94859 |
| 116 | 3 | 2564.986 | 1009.904 | 1.687083 | 0.036337 | True | 2.998516 | 99.95052 |
| 121 | 10 | 6221.373 | 2529.277 | 1.693735 | 0.036471 | True | 10.97393 | 109.7393 |
| 126 | 50 | 31990.54 | 12452.47 | 1.692059 | 0.038525 | True | 63.61878 | 127.2376 |
| 131 | 200 | 132594.2 | 53849.03 | 1.688984 | 0.036583 | True | 304.2652 | 152.1326 |
| 136 | N/A | N/A | N/A | N/A | N/A | True | N/A | N/A |

[symbol]

indicates data missing or illegible when filed

TABLE 12
The coefficients of variation for Lyso-Gb1.

| Lyso-Gb1 - CV % | 100% MeOH | 50% DMSO - 50% MeOH |
|---|---|---|
| QC 1 | 17.1% | 10.3% |
| QC 2 | 2.52% | 6.4% |
| QC 3 | 2.44% | 6.3% |
| QC 4 | 4.27% | 7% |
| QC 5 | 5.27% | 7% |

The cutoff for these CV % (inter-assay precision) according to the international CLSI guidelines is <15%.

TABLE 13
The coefficients of variation for Lyso-Gb3.

| Lyso-Gb3 - CV % | 100% MeOH | 50% DMSO - 50% MeOH |
|---|---|---|
| QC 1 | 34.94% | 14.5% |
| QC 2 | 19.88% | 12.5% |
| QC 3 | 5.93% | 11.4% |
| QC 4 | 10.51% | 6.3% |
| QC 5 | 8.31% | 6.3% |

The cutoff for these CV % (inter-assay precision) according to the international CLSI guidelines is <15%.

It can be concluded that the extraction solution described herein and hence the method subject of the invention is the only means which allows sensitive and robust detection of both Lyso-Gb1 and Lyso-Gb3 by a single sample processing step and by using a single detection step.

### Example 3. Establishment of Lyso-Gb1 and Lyso-GB3 Reference Ranges

Since the present study provides the first protocol to enable the detection of lysosomal storage disease biomarkers from a dried blood sample, no universal decision limits exist that can be used in clinical practice.

Samples for the reference range calculation were collected in the University Hospital in Antwerp, Belgium. Collection took place over a period of 3 years, from 2020 until 2022. A posteriori selection process was performed to collect nonclinical indicated blood samples (Ianni et al., Arch Pathol Lab Med, 2021). EDTA-anticoagulated blood samples from 1480 anonymous individuals in which the corresponding beta-glucocerebrosidase (GD) and alfa-galactosidase (FD) enzymatic activity were normal, were collected. Blood samples were spotted on filter paper and air dried at room temperature for at least 12 Hours. The DBS was sealed in a plastic bag and stored at −20° C. until further analysis. A minimum of 120 samples for each specific age group by gender was adhered, following literature and the CLSI guideline EP28-A3C, to establish age and gender-related reference ranges (Katayev et al., Am. J. Clin. Pathol., 2010; Horowitz et al., approved guideline-third edition. CLSI document EP28-A3c, 2010). Age categories were defined as 0-4:4-12:12-18:18-40; 40-60 and above 60 years old. Calculation of the parametric central 95 percent was performed according the CLSI EP28 A3CE guidelines, using the statistical program MedCalc Statistical Software (MedCalc Software Ltd., Ostend, Belgium). Data were Gaussian distributed using logarithmic mathematical transformation for Lyso-Gb1 and square transformation for Lyso-Gb3 in combination with a box-cox transformation. One round of outliner detection according to Tuckey test was performed and excluded. The gender and age related subgroups were compared based on the Z scores, described in the CLSI EP28 A3CE guideline (Harris and Boyd, Clin. Chem., 1990).

Gaussian distribution was achieved. Exclusion of the outliers resulted in 1370 remaining samples for Lyso-Gb1 and 1419 for Lyso-Gb3. The 95% reference range reference intervals are shown in Table 14.

TABLE 14
Identified reference ranges

| | | |
|---|---|---|
| Age in | Lyso-Gb1 (ng/ml) | Lyso-Gb3 (ng/ml) |

| | | | | |
|---|---|---|---|---|
| years | Female | Male | Female | Male |

| | | | |
|---|---|---|---|
| 0-4 | 0.282-6.890 | 0.0441-1.518 | 0.057-1.952 |
| 4-12 | 0.235-5.409 | 0.262-2.217 | 0.223-2.326 |

| | | |
|---|---|---|
| 12-18 | 0.237-6.385 | 0.138-2.014 |
| 18-40 | 0.272-7.111 | 0.145-1.971 |
| 40-60 | 0.319-6.392 | 0.085-2.014 |
| >60 | 0.317-4.928 | 0.118-1.774 |

The table contains healthy reference ranges, determined by the 2.5th and 97.5th percentile of the obtained measurements. Measurements are expressed in ng/ml (or 10-6 g/L).

By obtaining clinical statistical relevant reference ranges intervals based on a large number of enzymatic normal samples, conclusions about the most accurate cutoff limit values, useful for clinical practice diagnostics and follow-up under treatment, can be made. These reference ranges are essential in order to improve and facilitate the entire diagnostic work up and enable treatment in an earlier stage of disease and to closer follow-up efficacy of treatment.

1. An in vitro method for detecting multiple biomarkers indicative for lysosomal storage diseases, wherein the method comprises:
i) an extraction step of a dried blood spot sample or a portion thereof in an extraction solution comprising 35% to 65% dimethyl sulfoxide (DMSO) and 35% to 65% methanol (v/v);
ii) a centrifugation step comprising centrifugation of the sample obtained in step i) and collecting the supernatant (v/v);
iii) a detection step comprising detection of at least two biomarkers in the supernatant of step ii) each indicative for a distinct lysosomal storage disease.

2. The in vitro method according to claim 1, wherein the at least two biomarkers are or comprise sphingolipids, preferably wherein the at least two biomarkers comprise the glucosylsphingosine (Lyso-Gb1) biomarker and globotriaosylsphingosine (Lyso-Gb3) biomarker.

3. The in vitro method according to claim 1 or 2, wherein the extraction step comprises incubating the dried blood spot sample in the extraction solution for at least 10 minutes, preferably for at least 15 minutes, more preferably for at least 20 minutes.

4. The in vitro method according to any one of claims 1 to 3, wherein the extraction step is performed at a temperature of from 18° C. to 45° C., preferably at a temperature of from 25° C. to 40° C., more preferably at a temperature of about 37° C.

5. The in vitro method according to any one of claims 1 to 4, wherein the centrifugation step comprises centrifugation of the sample obtained in step i) for at least 5 minutes, preferably for at least 7.5 minutes, more preferably for at least 10 minutes.

6. The in vitro method according to any one of claims 1 to 5, wherein the sample obtained in step i) is centrifuged at from 2500 g to 15000 g, preferably at from 4000 g to 7500 g, more preferably at about 5250 g.

7. The in vitro method according to any one of claims 1 to 6, wherein the extraction solution used in step i) further comprises an internal standard, preferably wherein the extraction solution used in step i) further comprises an internal standard that comprises one or more stable isotope labeled reference counterpart of the biomarkers to be detected.

8. The in vitro method according to any one of claims 1 to 7, wherein the method is a method for detecting Gaucher disease and Fabry disease, more preferably wherein the method is indicative for Gaucher disease, Fabry disease, and acid sphingomyelinase deficiency.

9. The in vitro method according to any one of claims 1 to 8, wherein the at least two biomarkers comprise the glucosylsphingosine (Lyso-Gb1) biomarker, globotriaosylsphingosine (Lyso-Gb3) biomarker, and the lysosphingomyelin (Lyso-SPM) biomarker.

10. The in vitro method according to any one of claims 1 to 9, wherein the detection step comprises detection of at least one biomarker by mass spectrometry, preferably by tandem mass spectrometry, more preferably wherein the detection step comprises detection of at least two biomarkers by tandem mass spectrometry.

11. The in vitro method according to claim 10, wherein the detection step comprises a targeted tandem mass spectrometry assay, preferably a targeted tandem mass spectrometry assay selected from the group consisting of Selected Reaction Monitoring (SRM), Multiple Reaction Monitoring (MRM), or Parallel Reaction Monitoring (PRM).

12. The in vitro method according to claim 11, wherein the targeted mass spectrometry assay comprises measurement of a parent Lyso-Gb1 ion and a parent Lyso-Gb3 ion, preferably wherein the targeted mass spectrometry assay comprises measurement of a parent Lyso-Gb1 ion at 462.2 to 462.3 m/z and a parent Lyso-Gb3 ion at 786.2 to 786.5 m/z.

13. The in vitro method according to any one of claims 1 to 12, wherein the method further comprises a step of detecting a defective enzyme causative of a lysosomal storage disease and/or a reduction or absence of enzymatic activity of an enzyme causative of a lysosomal storage disease in the sample.

14. A kit of parts for detecting multiple biomarkers indicative for lysosomal storage diseases from a dried blood spot sample, wherein the kit comprises:
an extraction solution comprising 35% to 65% DMSO and 35% to 65% methanol (v/v);
an internal standard comprising at least two stable isotope labeled reference counterparts of said multiple biomarkers; and
a set of instructions for performing the method according to any one of claims 1 to 13.

15. The kit of parts according to claim 14, wherein the internal standard comprises as first reference biomarker stable isotope labeled Lyso-Gb1 and as second reference biomarker stable isotope labeled Lyso-Gb3.
